(19) United States
(12) Patent Application Publication
(10) Pub. No.: US 2025/0025568 A1
(43) Pub. Date: Jan. 23, 2025

(54) COMBINATION OF ANTIBODY-DRUG CONJUGATE AND PARP1 SELECTIVE INHIBITOR

(71) Applicants: AstraZeneca UK Limited, Cambridge (GB); DAIICHI SANKYO COMPANY, LIMITED, Tokyo (JP)

(72) Inventors: Matthew Simon SUNG, Wilmington, DE (US); Jerome Thomas METTETAL, Wilmington, DE (US); Elisabetta LEO, Cambridge (GB); Yann WALLEZ, Cambridge (GB); Theresa Angela PROIA, Wilmington, DE (US)

(73) Assignees: AstraZeneca UK Limited, Cambridge (GB); DAIICHI SANKYO COMPANY, LIMITED, Tokyo (JP)

(21) Appl. No.: 18/711,411

(22) PCT Filed: Nov. 17, 2022
(86) PCT No.: PCT/IB2022/061095

§ 371 Date: May 17, 2024

Related U.S. Application Data
(60) Provisional application No. 63/280,682, filed on Nov. 18, 2021.

(51) Int. Cl. A61K 47/68; A61K 31/496; A61P 35/00; C07K 16/30

(52) U.S. Cl. CPC A61K 47/68037; A61K 31/496; A61K 47/6851; A61P 35/00; C07K 16/30

(57) ABSTRACT

A pharmaceutical product for administration of an anti-TROP2 antibody-drug conjugate in combination with a PARP1 selective inhibitor is provided. The anti-TROP2 antibody-drug conjugate is an antibody-drug conjugate in which a drug-linker represented by the following formula (wherein A represents the connecting position to an anti-TROP2 antibody) is conjugated to an anti-TROP2 antibody via a thioether bond. Also provided is a therapeutic use and method wherein the anti-TROP2 antibody-drug conjugate and the PARP1 selective inhibitor are administered in combination to a subject: Formula (II)

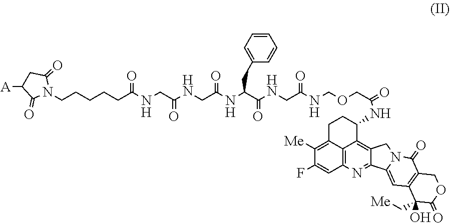

### TECHNICAL FIELD

The present disclosure relates to a pharmaceutical product for administration of a specific antibody-drug conjugate, having an antitumor drug conjugated to an anti-TROP2 antibody via a linker structure, in combination with a PARP1 selective inhibitor, and to a therapeutic use and method wherein the specific antibody-drug conjugate and the PARP1 selective inhibitor are administered in combination to a subject.

### BACKGROUND

The Poly (ADP-ribose) polymerase (PARP) family of enzymes plays an important role in a number of cellular processes, such as replication, recombination, chromatin remodeling, and DNA damage repair (O'Connor M J, Mol Cell (2015) 60 (4), 547-560). Single-strand break and double-strand break, as types of DNA damage, each have a repair mechanism. If the type of DNA damage is single-strand break, it will be repaired through base excision repair predominantly by PARP (poly[adenosine-5′-diphosphate (ADP)-ribose]polymerase) acting thereon. If the type of DNA damage is double-strand break, it will be repaired through homologous recombination repair predominantly by BRCA, ATM, RAD51, and the like, acting thereon (Lord C J, et al., Nature (2012) 481, 287-294).

PARP1 and PARP2 are the most extensively studied PARPs for their role in DNA damage repair. PARP1 is activated by DNA damage breaks and functions to catalyse the addition of poly (ADP-ribose) (PAR) chains to target proteins. This post-translational modification, known as PARylation, mediates the recruitment of additional DNA repair factors to DNA lesions. Following completion of this recruitment role, PARP auto-PARylation triggers the release of bound PARP from DNA to allow access to other DNA repair proteins to complete repair. Thus, the binding of PARP to damaged sites, its catalytic activity, and its eventual release from DNA are all important steps for a cancer cell to respond to DNA damage caused by chemotherapeutic agents and radiation therapy (Bai P., Mol Cell (2015) 58, 947-958).

Inhibition of PARP family enzymes has been exploited as a strategy to selectively kill cancer cells by inactivating complementary DNA repair pathways. A number of pre-clinical and clinical studies have demonstrated that tumor cells bearing deleterious alterations of BRCA1 or BRCA2, key tumor suppressor proteins involved in double-strand DNA break (DSB) repair by homologous recombination (HR), are selectively sensitive to small molecule inhibitors of the PARP family of DNA repair enzymes. Such tumors have deficient homologous recombination repair (HRR) pathways and are dependent on PARP enzymes function for survival. Although PARP inhibitor therapy has predominantly targeted BRCA-mutated cancers, PARP inhibitors have been tested clinically in non-BRCA-mutant tumors, those which exhibit homologous recombination deficiency (HRD) (Turner N, Tutt A, Ashworth A. Hallmarks of ‘BRCAness’ in sporadic cancers. Nat Rev Cancer 2004; 4:814-9).

PARP inhibitors are drugs that have the function of inhibiting PARP (particularly PARP1 and PARP2), and thus preventing single-strand break repair. Some cancers including breast cancer and ovarian cancer are known to have an abnormality in double-strand break repair, and PARP inhibitors have been revealed to have antitumor effects due to synthetic lethality against these cancers (Benafif S, et al., Onco. Targets Ther. (2015) 8, 519-528; Fong P C, et al., N. Engl. J. Med. (2009) 361, 123-134; Fong P C, et al., J. Clin. Oncol. (2010) 28, 2512-2519; Gelmon K A, et al., Lancet Oncol. (2011) 12, 852-861).

Examples of PARP inhibitors and their mechanism of action are taught in e.g. WO 2004/080976. Known PARP inhibitors include olaparib (Menear K A, et al., J. Med. Chem. (2008) 51, 6581-6591), rucaparib (Gillmore A T, et al., Org. Process Res. Dev. (2012) 16, 1897-1904), niraparib (Jones P, et al., J. Med. Chem. (2009) 52, 7170-7185), and talazoparib (Shen Y, et al., Clin. Cancer Res. (2013) 19 (18), 5003-15).

It is believed that PARP inhibitors having improved selectivity for PARP1 may possess improved efficacy and reduced toxicity compared to non-selective PARP inhibitors. It is believed also that selective strong inhibition of PARP1 would lead to trapping of PARP1 on DNA, resulting in DNA double-strand breaks (DSBs) through collapse of replication forks in S-phase. It is believed also that PARP1-DNA trapping is an effective mechanism for selectively killing tumor cells having HRD.

Antibody-drug conjugates (ADCs), which are composed of a cytotoxic drug conjugated to an antibody, can deliver the drug selectively to cancer cells and can thus be expected to cause accumulation of the drug within cancer cells and to kill the cancer cells (Ducry, L., et al., Bioconjugate Chem. (2010) 21, 5-13; Alley, S. C., et al., Current Opinion in Chemical Biology (2010) 14, 529-537; Damle N. K. Expert Opin. Biol. Ther. (2004) 4, 1445-1452; Senter P. D., et al., Nature Biotechnology (2012) 30, 631-637; Burris H A., et al., J. Clin. Oncol. (2011) 29 (4): 398-405).

One such antibody-drug conjugate is datopotamab deruxtecan, which is composed of a TROP2-targeting antibody and a derivative of exatecan. In particular, WO 2015/098099 and WO 2020/240467 provide detailed descriptions of exemplary TROP2-targeting antibody-drug conjugates, including datopotamab deruxtecan (DS-1062). Datopotamab deruxtecan has shown clinical efficacy in multiple tumor types, including lung cancer and breast cancer. There is a need to identify combination partners for anti-TROP2 antibody-drug conjugates, such as datopotamab deruxtecan, to enhance efficacy, increase durability of therapeutic response, improve tolerance to patients and/or reduce dose-dependent toxicity.

Despite the therapeutic potential of anti-TROP2 antibody-drug conjugates such as datopotamab deruxtecan (DS-1062), of PARP inhibitors such as PARP1 selective inhibitors, and of antibody-drug conjugates in combination with olaparib or other PARP inhibitors (WO 2020/122034), no literature is published that describes a test result demonstrating an excellent effect of combined use of a TROP2-targeting antibody-drug conjugate and a PARP1 selective inhibitor.

Accordingly, a need remains for improved therapeutic compositions and methods, that can enhance efficacy of existing cancer treating agents, increase durability of therapeutic response, improve tolerance to patients and/or reduce dose-dependent toxicity.

### SUMMARY OF DISCLOSURE

The antibody-drug conjugate used in the present disclosure (an anti-TROP2 antibody-drug conjugate that includes a derivative of the topoisomerase I inhibitor exatecan, as a component) has been confirmed to exhibit an excellent antitumor effect in the treatment of certain cancers such as breast cancer and lung cancer, when administered singly. Furthermore, a PARP1 inhibitor has been confirmed to exhibit an antitumor effect in the treatment of certain cancers. However, it is desired to provide a medicine and treatment which can obtain a superior antitumor effect in the treatment of cancers, such as enhanced efficacy, increased durability of therapeutic response and/or reduced dose-dependent toxicity.

The present disclosure provides a pharmaceutical product which can exhibit an excellent antitumor effect in the treatment of cancers, through administration of an anti-TROP2 antibody-drug conjugate in combination with a PARP1 selective inhibitor. The present disclosure also provides a therapeutic use and method wherein the anti-TROP2 antibody-drug conjugate and PARP1 selective inhibitor are administered in combination to a subject.

Specifically, the present disclosure relates to the following [1] to:

[1] a pharmaceutical product comprising an anti-TROP2 antibody-drug conjugate and a PARP1 selective inhibitor for administration in combination, wherein the anti-TROP2 antibody-drug conjugate is an antibody-drug conjugate in which a drug-linker represented by the following formula:

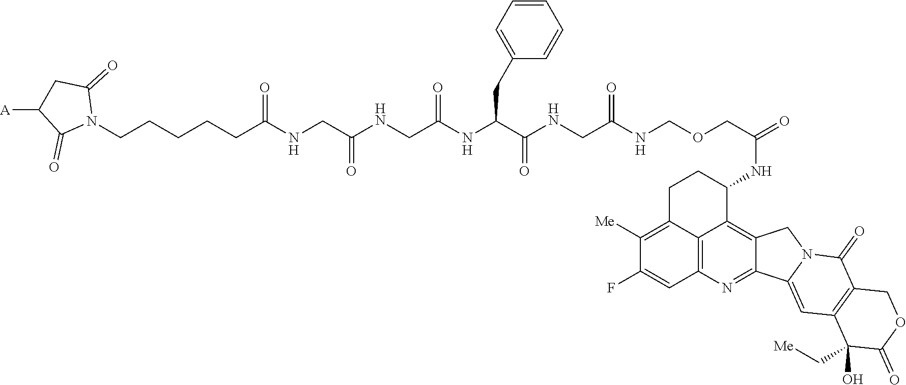

wherein A represents the connecting position to an antibody, is conjugated to an anti-TROP2 antibody via a thioether bond;

[2] the pharmaceutical product according to [1], wherein the PARP1 selective inhibitor is a compound represented by the following formula (I):

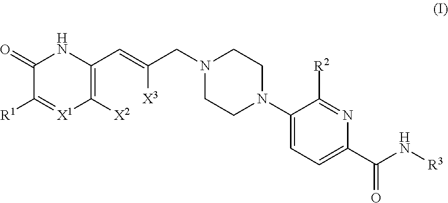

wherein:
X¹ and X² are each independently selected from N and C(H),
X³ is independently selected from N and C(R⁴), wherein R⁴ is H or fluoro,
R¹ is C₁₋₄ alkyl or C₁₋₄ fluoroalkyl,
R² is independently selected from H, halo, C₁₋₄ alkyl, and C₁₋₄ fluoroalkyl, and
R³ is H or C₁₋₄ alkyl,
or a pharmaceutically acceptable salt thereof
provided that:
when X¹ is N, then X² is C(H), and X³ is C(R⁴),
when X² is N, then X¹═C(H), and X³ is C(R⁴), and
when X³ is N, then X¹ and X² are both C(H);

[3] the pharmaceutical product according to [2] wherein, in formula (I), R³ is C₁₋₄ alkyl;

[4] the pharmaceutical product according to [3] wherein, in formula (I), R³ is methyl;

[5] the pharmaceutical product according to any one of [2] to [4] wherein, in formula (I), R¹ is ethyl;

[6] the pharmaceutical product according to [1], wherein the PARP1 selective inhibitor is a compound represented by the following formula (Ia):

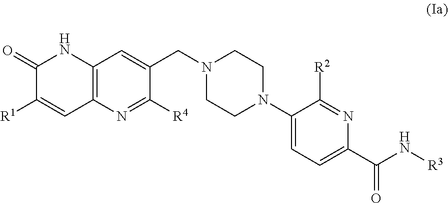

wherein
R¹ is C₁₋₄ alkyl,
R² is selected from H, halo, C₁₋₄ alkyl, and C₁₋₄ fluoroalkyl,
R³ is H or C₁₋₄ alkyl, and
R⁴ is H,
or a pharmaceutically acceptable salt thereof;

[7] the pharmaceutical product according to [6] wherein, in formula (Ia), R² is H or halo;

[8] the pharmaceutical product according to [6] wherein in formula (Ia), R¹ is ethyl, R² is selected from H, chloro and fluoro, and R³ is methyl;

[9] the pharmaceutical product according to [1], wherein the PARP1 selective inhibitor is AZD5305, also known as AZ14170049, represented by the following formula:

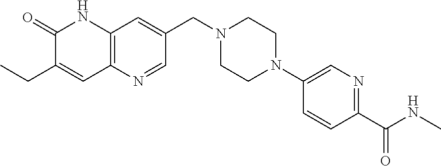 [SMILES: CCc1cc2ncc(CN3CCN(c4ccc(C(=O)NC)nc4)CC3)cc2[nH]c1=O]

or a pharmaceutically acceptable salt thereof;

[10] the pharmaceutical product according to any one of [1] to [9], wherein the anti-TROP2 antibody is an antibody comprising a heavy chain comprising CDRH1 consisting of an amino acid sequence represented by SEQ ID NO: 3 [=amino acid residues 50 to 54 of SEQ ID NO: 1], CDRH2 consisting of an amino acid sequence represented by SEQ ID NO: 4 [=amino acid residues 69 to 85 of SEQ ID NO: 1] and CDRH3 consisting of an amino acid sequence represented by SEQ ID NO: 5 [=amino acid residues 118 to 129 of SEQ ID NO: 1], and a light chain comprising CDRL1 consisting of an amino acid sequence represented by SEQ ID NO: 6 [=amino acid residues 44 to 54 of SEQ ID NO: 2], CDRL2 consisting of an amino acid sequence represented by SEQ ID NO: 7 [=amino acid residues 70 to 76 of SEQ ID NO: 2] and CDRL3 consisting of an amino acid sequence represented by SEQ ID NO: 8 [=amino acid residues 109 to 117 of SEQ ID NO: 2];

[11] the pharmaceutical product according to [10], wherein the anti-TROP2 antibody is an antibody comprising a heavy chain comprising a heavy chain variable region consisting of an amino acid sequence represented by SEQ ID NO: 9 [=amino acid residues 20 to 140 of SEQ ID NO: 1] and a light chain comprising a light chain variable region consisting of an amino acid sequence represented by SEQ ID NO: 10 [=amino acid residues 21 to 129 of SEQ ID NO: 2];

[12] the pharmaceutical product according to [11], wherein the anti-TROP2 antibody is an antibody comprising a heavy chain consisting of an amino acid sequence consisting of an amino acid sequence represented by SEQ ID NO: 12 [=amino acid residues 20 to 470 of SEQ ID NO: 1] and a light chain consisting of an amino acid sequence represented by SEQ ID NO: 13 [=amino acid residues 21 to 234 of SEQ ID NO: 2];

[13] the pharmaceutical product according to [11], wherein the anti-TROP2 antibody is an antibody comprising a heavy chain consisting of an amino acid sequence represented by SEQ ID NO: 11 [=amino acid residues 20 to 469 of SEQ ID NO: 1] and a light chain consisting of an amino acid sequence represented by SEQ ID NO: 13 [=amino acid residues 21 to 234 of SEQ ID NO: 2];

[14] the pharmaceutical product according to any one of [1] to [13], wherein the average number of units of the drug-linker conjugated per antibody molecule in the antibody-drug conjugate is in the range of from 2 to 8;

[15] the pharmaceutical product according to [14], wherein the average number of units of the drug-linker conjugated per antibody molecule in the antibody-drug conjugate is in the range of from 3.5 to 4.5;

[16] the pharmaceutical product according to [15], wherein the anti-TROP2 antibody-drug conjugate is datopotamab deruxtecan (DS-1062);

[17] the pharmaceutical product according to any one of [1] to wherein the product is a composition comprising the anti-TROP2 antibody-drug conjugate and the PARP1 selective inhibitor, for simultaneous administration;

[18] the pharmaceutical product according to any one of [1] to wherein the product is a combined preparation comprising the anti-TROP2 antibody-drug conjugate and the PARP1 selective inhibitor, for sequential or simultaneous administration;

[19] the pharmaceutical product according to any one of [1] to wherein the anti-TROP2 antibody-drug conjugate is to be administered at a dose of 6 mg/kg body weight;

[20] the pharmaceutical product according to wherein the dose of the anti-TROP2 antibody-drug conjugate is to be administered once every three weeks;

[21] the pharmaceutical product according to any one of [1] to wherein the PARP1 selective inhibitor is to be administered daily for the first week, second week, and/or third week of a three week cycle;

[22] the pharmaceutical product according to any one of [1] to [21], wherein the product is for treating cancer;

[23] the pharmaceutical product according to [22], wherein the cancer is at least one selected from the group consisting of breast cancer, lung cancer, colorectal cancer, gastric cancer, esophageal cancer, head-and-neck cancer, esophagogastric junction adenocarcinoma, biliary tract cancer, Paget's disease, pancreatic cancer, ovarian cancer, uterine carcinosarcoma, urothelial cancer, prostate cancer, bladder cancer, gastrointestinal stromal tumor, digestive tract stromal tumor, cervical cancer, squamous cell carcinoma, peritoneal cancer, liver cancer, hepatocellular cancer, corpus uteri carcinoma, kidney cancer, vulval cancer, thyroid cancer, penis cancer, leukemia, malignant lymphoma, plasmacytoma, myeloma, glioblastoma multiforme, osteosarcoma, sarcoma, endometrial cancer, and melanoma;

[24] the pharmaceutical product according to [23], wherein the cancer is breast cancer;

[25] the pharmaceutical product according to [24], wherein the breast cancer is triple negative breast cancer;

[26] the pharmaceutical product according to [24], wherein the breast cancer is hormone receptor (HR)-positive, HER2-negative breast cancer;

[27] the pharmaceutical product according to [23], wherein the cancer is lung cancer;

[28] the pharmaceutical product according to [27], wherein the lung cancer is non-small cell lung cancer;

[29] the pharmaceutical product according to [28], wherein the non-small cell lung cancer is non-small cell lung cancer with actionable genomic alterations;

[30] the pharmaceutical product according to [28], wherein the non-small cell lung cancer is non-small cell lung cancer lung cancer without actionable genomic alterations;

[31] the pharmaceutical product according to [27], wherein the lung cancer is small cell lung cancer;

[32] the pharmaceutical product according to [23], wherein the cancer is colorectal cancer;

[33] the pharmaceutical product according to [23], wherein the cancer is gastric cancer;

[34] the pharmaceutical product according to [23], wherein the cancer is pancreatic cancer;

[35] the pharmaceutical product according to [23], wherein the cancer is ovarian cancer;

[36] the pharmaceutical product according to [23], wherein the cancer is prostate cancer;

[37] the pharmaceutical product according to [23], wherein the cancer is kidney cancer;

[38] the pharmaceutical product according to [23], wherein the cancer is bladder cancer;

[39] the pharmaceutical product according to [23], wherein the cancer is biliary tract cancer;

[40] the pharmaceutical product according to [23], wherein the cancer is cervical cancer;

[41] the pharmaceutical product according to [23], wherein the cancer is endometrial cancer;

[42] the pharmaceutical product according to any one of [23] to [41], wherein the cancer is deficient in Homologous Recombination (HR) dependent DNA DSB repair activity;

[43] the pharmaceutical product according to any one of [23] to [41], wherein the cancer is not deficient in Homologous Recombination (HR) dependent DNA DSB repair activity;

[44] the pharmaceutical product according to any one of [23] to [41], wherein the cancer is exhibits resistance or refractoriness to a previous treatment with a PARP inhibitor;

[45] the pharmaceutical product according to [44], wherein the previous treatment is with a PARP inhibitor selected from olaparib, rucaparib, niraparib, talazoparib and veliparib;

[46] a pharmaceutical product as defined in any one of [1] to [21], for use in treating cancer;

[47] the pharmaceutical product for the use according to [46], wherein the cancer is as defined in any one of to [45];

[48] use of an anti-TROP2 antibody-drug conjugate or a PARP1 selective inhibitor in the manufacture of a medicament for administration of the anti-TROP2 antibody-drug conjugate and the PARP1 selective inhibitor in combination, wherein the anti-TROP2 antibody-drug conjugate and the PARP1 selective inhibitor are as defined in any one of [1] to [16], for treating cancer;

[49] the use according to [48], wherein the cancer is as defined in any one of to [45];

[50] the use according to or wherein the medicament is a composition comprising the anti-TROP2 antibody-drug conjugate and the PARP1 selective inhibitor, for simultaneous administration;

[51] the use according to or wherein the medicament is a combined preparation comprising the anti-TROP2 antibody-drug conjugate and the PARP1 selective inhibitor, for sequential or simultaneous administration;

[52] the use according to any one of to wherein the anti-TROP2 antibody-drug conjugate is to be administered at a dose of 6 mg/kg body weight;

[53] the use according to wherein the dose of the anti-TROP2 antibody-drug conjugate is to be administered once every three weeks;

[54] the use according to any one of to wherein the PARP1 selective inhibitor is to be administered daily for the first week, second week, and/or third week of a three week cycle;

[55] an anti-TROP2 antibody-drug conjugate for use, in combination with a PARP1 selective inhibitor, in the treatment of cancer, wherein the anti-TROP2 antibody-drug conjugate and the PARP1 selective inhibitor are as defined in any one of [1] to [16];

[56] the anti-TROP2 antibody-drug conjugate for the use according to [55], wherein the cancer is as defined in any one of to [45];

[57] the anti-TROP2 antibody-drug conjugate for the use according to or [56], wherein the use comprises administration of the anti-TROP2 antibody-drug conjugate and the PARP1 selective inhibitor sequentially;

[58] the anti-TROP2 antibody-drug conjugate for the use according to any one of to wherein the anti-TROP2 antibody-drug conjugate is to be administered at a dose of 6 mg/kg body weight;

[59] the anti-TROP2 antibody-drug conjugate for the use according to wherein the dose of the anti-TROP2 antibody-drug conjugate is to be administered once every three weeks;

[60] the anti-TROP2 antibody-drug conjugate for the use according to any one of to wherein the PARP1 selective inhibitor is to be administered daily for the first week, second week, and/or third week of a three week cycle;

[61] the anti-TROP2 antibody-drug conjugate for the use according to or [56], wherein the use comprises administration of the anti-TROP2 antibody-drug conjugate and the PARP1 selective inhibitor simultaneously;

[62] an anti-TROP2 antibody-drug conjugate for use in the treatment of cancer in a subject, wherein said treatment comprises the separate, sequential or simultaneous administration of i) said anti-TROP2 antibody-drug conjugate, and ii) a PARP1 selective inhibitor to said subject, wherein said anti-TROP2 antibody-drug conjugate and said PARP1 selective inhibitor are as defined in any one of [1] to [16];

[63] the anti-TROP2 antibody-drug conjugate for the use according to wherein the anti-TROP2 antibody-drug conjugate is to be administered at a dose of 6 mg/kg body weight;

[64] the anti-TROP2 antibody-drug conjugate for the use according to wherein the dose of the anti-TROP2 antibody-drug conjugate is to be administered once every three weeks;

[65] the anti-TROP2 antibody-drug conjugate for the use according to any one of to wherein the PARP1 selective inhibitor is to be administered daily for the first week, second week, and/or third week of a three week cycle;

[66] a PARP1 selective inhibitor for use, in combination with an anti-TROP2 antibody-drug conjugate, in the treatment of cancer, wherein the anti-TROP2 antibody-drug conjugate and the PARP1 selective inhibitor are as defined in any one of [1] to [16];

[67] the PARP1 selective inhibitor for the use according to [66], wherein the cancer is as defined in any one of to [45];

[68] the PARP1 selective inhibitor for the use according to or [67], wherein the use comprises administration of the anti-TROP2 antibody-drug conjugate and the PARP1 selective inhibitor sequentially;

[69] the PARP1 selective inhibitor for the use according to any one of to wherein the anti-TROP2 antibody-drug conjugate is to be administered at a dose of 6 mg/kg body weight;

[70] the PARP1 selective inhibitor for the use according to wherein the dose of the anti-TROP2 antibody-drug conjugate is to be administered once every three weeks;

[71] the PARP1 selective inhibitor for the use according to any one of to wherein the PARP1 selective inhibitor is to be administered daily for the first week, second week, and/or third week of a three week cycle;

[72] the PARP1 selective inhibitor for the use according to or [67], wherein the use comprises administration of the anti-TROP2 antibody-drug conjugate and the PARP1 selective inhibitor simultaneously;

[73] a PARP1 selective inhibitor for use in the treatment of cancer in a subject, wherein said treatment comprises the separate, sequential or simultaneous administration of i) said PARP1 selective inhibitor, and ii) an anti-TROP2 antibody-drug conjugate to said subject, wherein said PARP1 selective inhibitor and said anti-TROP2 antibody-drug conjugate are as defined in any one of [1] to [16];

[74] the PARP1 selective inhibitor for the use according to wherein the anti-TROP2 antibody-drug conjugate is to be administered at a dose of 6 mg/kg body weight;

[75] the PARP1 selective inhibitor for the use according to wherein the dose of the anti-TROP2 antibody-drug conjugate is to be administered once every three weeks;

[76] the PARP1 selective inhibitor for the use according to any one of to wherein the PARP1 selective inhibitor is to be administered daily for the first week, second week, and/or third week of a three week cycle;

[77] a method of treating cancer comprising administering an anti-TROP2 antibody-drug conjugate and a PARP1 selective inhibitor as defined in any one of [1] to [16] in combination to a subject in need thereof;

[78] the method according to [77], wherein the cancer is as defined in any one of to;

[79] the method according to or [78], wherein the method comprises administering the anti-TROP2 antibody-drug conjugate and the PARP1 selective inhibitor sequentially;

[80] the method according to any one of to wherein the method comprises administering the anti-TROP2 antibody-drug conjugate at a dose of 6 mg/kg body weight.

[81] the method according to wherein the method comprises administering the dose of the anti-TROP2 antibody-drug conjugate once every three weeks.

[82] the method according to any one of to wherein the method comprises administering the PARP1 selective inhibitor daily for the first week, second week, and/or third week of a three week cycle; and

[83] the method according to or [78], wherein the method comprises administering the anti-TROP2 antibody-drug conjugate and the PARP1 selective inhibitor simultaneously.

### Advantageous Effects of Disclosure

The present disclosure provides a pharmaceutical product wherein an anti-TROP2 antibody-drug conjugate, having an antitumor drug conjugated to an anti-TROP2 antibody via a linker structure, and a PARP1 selective inhibitor are administered in combination, and a therapeutic use and method wherein the anti-TROP2 antibody-drug conjugate and the PARP1 selective inhibitor are administered in combination to a subject. Thus, the present disclosure can provide a medicine and treatment which can obtain a superior antitumor effect in the treatment of cancers.

### BRIEF DESCRIPTION OF DRAWINGS

FIG. 1 is a diagram showing the amino acid sequence of a heavy chain of an anti-TROP2 antibody (SEQ ID NO: 1).

FIG. 2 is a diagram showing the amino acid sequence of a light chain of an anti-TROP2 antibody (SEQ ID NO: 2).

FIG. 3 is a diagram showing the amino acid sequence of a heavy chain CDRH1 (SEQ ID NO: 3 [=amino acid residues 50 to 54 of SEQ ID NO: 1]).

FIG. 4 is a diagram showing the amino acid sequence of a heavy chain CDRH2 (SEQ ID NO: 4 [=amino acid residues 69 to 85 of SEQ ID NO: 1]).

FIG. 5 is a diagram showing the amino acid sequence of a heavy chain CDRH3 (SEQ ID NO: 5 [=amino acid residues 118 to 129 of SEQ ID NO: 1]).

FIG. 6 is a diagram showing the amino acid sequence of a light chain CDRL1 (SEQ ID NO: 6 [=amino acid residues 44 to 54 of SEQ ID NO: 2]).

FIG. 7 is a diagram showing the amino acid sequence of a light chain CDRL2 (SEQ ID NO: 7 [=amino acid residues 70 to 76 of SEQ ID NO: 2]).

FIG. 8 is a diagram showing the amino acid sequence of a light chain CDRL3 (SEQ ID NO: 8 [=amino acid residues 109 to 117 of SEQ ID NO: 2]).

FIG. 9 is a diagram showing the amino acid sequence of a heavy chain variable region (SEQ ID NO: 9 [=amino acid residues 20 to 140 of SEQ ID NO: 1]).

FIG. 10 is a diagram showing the amino acid sequence of a light chain variable region (SEQ ID NO: 10 [=amino acid residues 21 to 129 of SEQ ID NO: 2]).

FIG. 11 is a diagram showing the amino acid sequence of a heavy chain (SEQ ID NO: 11 [=amino acid residues 20 to 469 of SEQ ID NO: 1]).

FIGS. 12A and 12B are diagrams showing combination matrices obtained with high-throughput screens combining DS-1062 with AZD5305 (PARP1 selective inhibitor) in TROP2-expressing lung cancer cell lines.

FIGS. 13A and 13B are diagrams showing combination matrices obtained with high-throughput screens combining DS-1062 with AZD5305 in TROP2-expressing breast cancer cell lines.

FIG. 14 is a diagram showing combination Emax and Loewe synergy scores in cell lines treated with DS-1062 combined with AZD5305.

FIGS. 15A and 15B are diagrams showing combination matrices obtained with high-throughput screens combining DS-1062 with olaparib in TROP2-expressing lung cancer cell lines.

FIGS. 16A and 16B are diagrams showing combination matrices obtained with high-throughput screens combining DS-1062 with olaparib in TROP2-expressing breast cancer cell lines.

FIG. 17 is a diagram showing combination Emax and Loewe synergy scores in cell lines treated with DS-1062 combined with olaparib.

FIG. 18 is a graph showing tumor volumes for in vivo treatments with DS-1062 or AZD5305 alone or with DS-1062 in combination with AZD5305.

FIG. 19A is a graph showing tumor volumes for in vivo treatments with DS-1062 or AZD5305 alone or with DS-1062 in combination with AZD5305. The dotted line represents the end of the AZD5305 dosing period. FIG. 19B is a graph of percentage of mice achieving a complete response in each treatment group from this study.

FIG. 20 is a graph showing tumor volumes for in vivo treatments with DS-1062 or AZD5305 alone or with DS-1062 in combination with AZD5305, in a NCI-N87 xenograft model.

FIG. 21 is a graph showing tumor volumes for in vivo treatments with DS-1062 or AZD5305 alone or with DS-1062 in combination with AZD5305, in a NCI-N87 xenograft.

FIG. 22 is a graph showing tumor volumes for in vivo treatments with DS-1062 or AZD5305 alone or with DS-1062 in combination with AZD5305, in a CTG-3718 xenograft model.

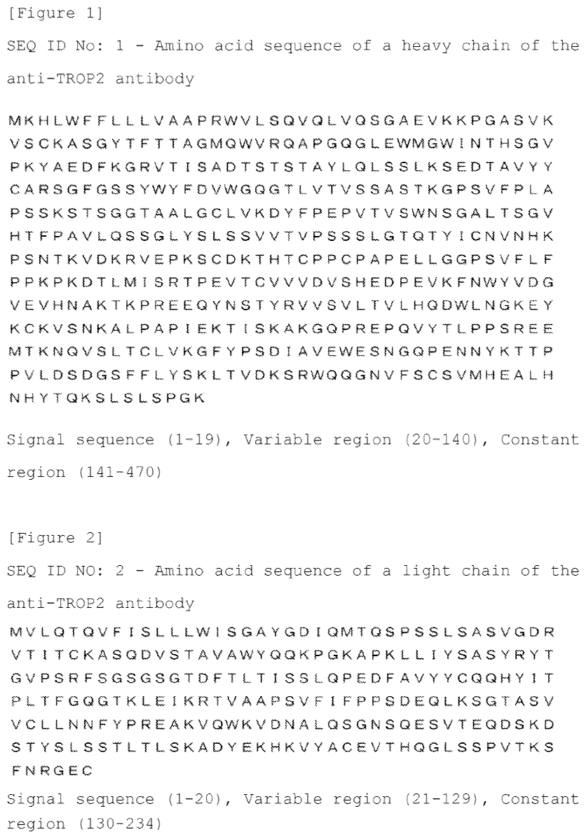

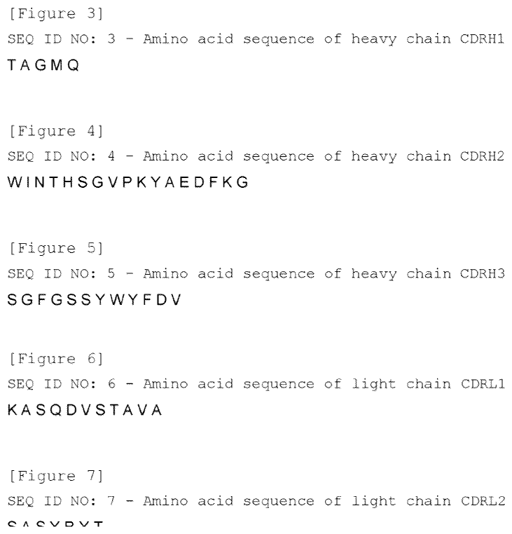

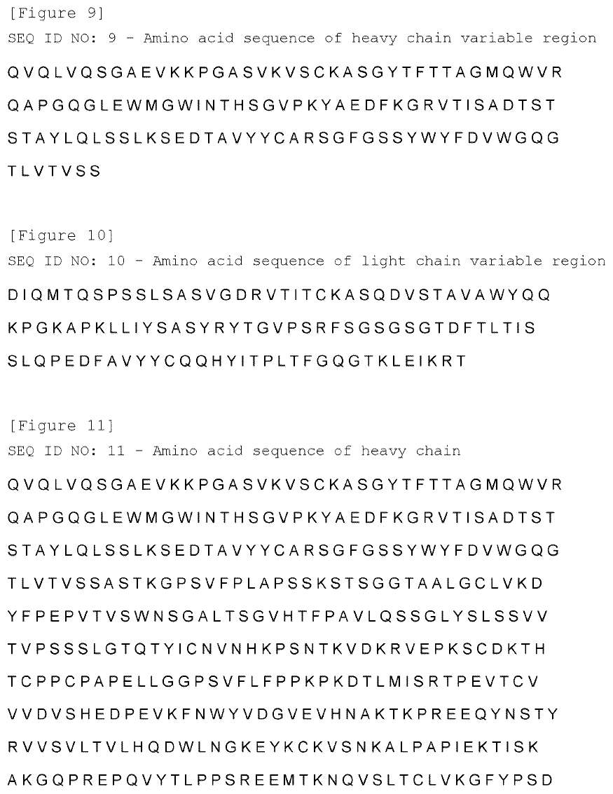

In order that the present disclosure can be more readily understood, certain terms are first defined. Additional definitions are set forth throughout the detailed description.

Before describing the present disclosure in detail, it is to be understood that this disclosure is not limited to specific compositions or method steps, as such can vary. As used in this specification and the appended claims, the singular forms “a”, “an” and “the” include plural referents unless the context clearly dictates otherwise. The terms “a” (or “an”), as well as the terms “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range.

It is understood that wherever aspects are described herein with the language “comprising”, otherwise analogous aspects described in terms of “consisting of” and/or “consisting essentially of” are also provided.

The terms “inhibit”, “block”, and “suppress” are used interchangeably herein and refer to any statistically significant decrease in biological activity, including full blocking of the activity. For example, “inhibition” can refer to a decrease of about 10%, 20%, 30%, 40%, 50%, 60%, 70%, 80%, 90% or 100% in biological activity. Cellular proliferation can be assayed using art recognized techniques which measure rate of cell division, and/or the fraction of cells within a cell population undergoing cell division, and/or rate of cell loss from a cell population due to terminal differentiation or cell death (e.g., thymidine incorporation).

The term “subject” refers to any animal (e.g., a mammal), including, but not limited to humans, non-human primates, rodents, and the like, which is to be the recipient of a particular treatment. Typically, the terms “subject” and “patient” are used interchangeably herein in reference to a human subject.

The term “pharmaceutical product” refers to a preparation which is in such form as to permit the biological activity of the active ingredients, either as a composition containing all the active ingredients (for simultaneous administration), or as a combination of separate compositions (a combined preparation) each containing at least one but not all of the active ingredients (for administration sequentially or simultaneously), and which contains no additional components which are unacceptably toxic to a subject to which the product would be administered. Such product can be sterile. By “simultaneous administration” is meant that the active ingredients are administered at the same time. By “sequential administration” is meant that the active ingredients are administered one after the other, in either order, at a time interval between the individual administrations. The time interval can be, for example, less than 24 hours, preferably less than 6 hours, more preferably less than 2 hours.

Terms such as “treating” or “treatment” or “to treat” or “alleviating” or “to alleviate” refer to both (1) therapeutic measures that cure, slow down, lessen symptoms of, and/or halt progression of a diagnosed pathologic condition or disorder and (2) prophylactic or preventative measures that prevent and/or slow the development of a targeted pathologic condition or disorder. Thus, those in need of treatment include those already with the disorder; those prone to have the disorder; and those in whom the disorder is to be prevented. In certain aspects, a subject is successfully “treated” for cancer according to the methods of the present disclosure if the patient shows, e.g., total, partial, or transient remission of a certain type of cancer.

The terms “cancer”, “tumor”, “cancerous”, and “malignant” refer to or describe the physiological condition in mammals that is typically characterized by unregulated cell growth. Examples of cancers include but are not limited to, breast cancer, lung cancer, colorectal cancer, gastric cancer, esophageal cancer, head-and-neck cancer, esophagogastric junction adenocarcinoma, biliary tract cancer, Paget's disease, pancreatic cancer, ovarian cancer, uterine carcinosarcoma, urothelial cancer, prostate cancer, bladder cancer, gastrointestinal stromal tumor, digestive tract stromal tumor, cervical cancer, squamous cell carcinoma, peritoneal cancer, liver cancer, hepatocellular cancer, corpus uteri carcinoma, kidney cancer, vulval cancer, thyroid cancer, penis cancer, leukemia, malignant lymphoma, plasmacytoma, myeloma, glioblastoma multiforme, osteosarcoma, sarcoma, endometrial cancer, and melanoma. Cancers include hematological malignancies such as acute myeloid leukemia, multiple myeloma, chronic lymphocytic leukemia, diffuse large B cell lymphoma, Burkitt's lymphoma, follicular lymphoma and solid tumors such as breast cancer, lung cancer, neuroblastoma and colon cancer.

The term “cytotoxic agent” as used herein is defined broadly and refers to a substance that inhibits or prevents the function of cells and/or causes destruction of cells (cell death), and/or exerts anti-neoplastic/anti-proliferative effects. For example, a cytotoxic agent prevents directly or indirectly the development, maturation, or spread of neoplastic tumor cells. The term includes also such agents that cause a cytostatic effect only and not a mere cytotoxic effect. The term includes chemotherapeutic agents as specified below, as well as other TROP2 antagonists, anti-angiogenic agents, tyrosine kinase inhibitors, protein kinase A inhibitors, members of the cytokine family, radioactive isotopes, and toxins such as enzymatically active toxins of bacterial, fungal, plant or animal origin.

The term “chemotherapeutic agent” is a subset of the term “cytotoxic agent” comprising natural or synthetic chemical compounds.

In accordance with the methods or uses of the present disclosure, compounds of the present disclosure may be administered to a patient to promote a positive therapeutic response with respect to cancer. The term “positive therapeutic response” with respect to cancer treatment refers to an improvement in the symptoms associated with the disease. For example, an improvement in the disease can be characterized as a complete response. The term “complete response” refers to an absence of clinically detectable disease with normalization of any previous test results. Alternatively, an improvement in the disease can be categorized as being a partial response. A “positive therapeutic response” encompasses a reduction or inhibition of the progression and/or duration of cancer, the reduction or amelioration of the severity of cancer, and/or the amelioration of one or more symptoms thereof resulting from the administration of compounds of the present disclosure. In specific aspects, such terms refer to one, two or three or more results following the administration of compounds of the instant disclosure:
(1) a stabilization, reduction or elimination of the cancer cell population;
(2) a stabilization or reduction in cancer growth;
(3) an impairment in the formation of cancer;
(4) eradication, removal, or control of primary, regional and/or metastatic cancer;
(5) a reduction in mortality;
(6) an increase in disease-free, relapse-free, progression-free, and/or overall survival, duration, or rate;
(7) an increase in the response rate, the durability of response, or number of patients who respond or are in remission;
(8) a decrease in hospitalization rate,
(9) a decrease in hospitalization lengths,
(10) the size of the cancer is maintained and does not increase or increases by less than 10%, preferably less than 5%, preferably less than 4%, preferably less than 2%, and
(11) an increase in the number of patients in remission.
(12) a decrease in the number of adjuvant therapies (e.g., chemotherapy or hormonal therapy) that would otherwise be required to treat the cancer.

Clinical response can be assessed using screening techniques such as PET, magnetic resonance imaging (MRI) scan, x-radiographic imaging, computed tomographic (CT) scan, flow cytometry or fluorescence-activated cell sorter (FACS) analysis, histology, gross pathology, and blood chemistry, including but not limited to changes detectable by ELISA, RIA, chromatography, and the like. In addition to these positive therapeutic responses, the subject undergoing therapy can experience the beneficial effect of an improvement in the symptoms associated with the disease.

Alkyl groups and moieties are straight or branched chain, e.g. C₁₋₈ alkyl, C₁₋₆ alkyl, C₁₋₄ alkyl or C₅₋₆ alkyl. Examples of alkyl groups are methyl, ethyl, n-propyl, iso-propyl, n-butyl, t-butyl, n-pentyl, n-hexyl, n-heptyl and n-octyl, such as methyl or n-hexyl.

Fluoroalkyl groups are alkyl groups in which one or more H atoms is replaced with one or more fluoro atoms, e.g. C₁₋₈ fluoroalkyl, C₁₋₆ fluoroalkyl, C₁₋₄ fluoroalkyl or C₅₋₆ fluoroalkyl. Examples include fluoromethyl (CH₂F—), difluromethyl (CHF₂—), trifluoromethyl (CF₃—), 2,2,2-trifluoroethyl (CF₃CH₂—), 1,1-difluoroethyl (CH₃CHF₂—), 2,2-difluoroethyl (CHF₂CH₂—), and 2-fluoroethyl (CH₂FCH₂—).

Halo means fluoro, chloro, bromo, and iodo. In an embodiment, halo is fluoro or chloro.

As used herein, the phrase “effective amount” means an amount of a compound or composition which is sufficient enough to significantly and positively modify the symptoms and/or conditions to be treated (e.g., provide a positive clinical response). The effective amount of an active ingredient for use in a pharmaceutical product will vary with the particular condition being treated, the severity of the condition, the duration of the treatment, the nature of concurrent therapy, the particular active ingredient(s) being employed, the particular pharmaceutically-acceptable excipient(s)/carrier(s) utilized, and like factors within the knowledge and expertise of the attending physician. In particular, an effective amount of a compound for use in the treatment of cancer in combination with the antibody-drug conjugate is an amount such that the combination is sufficient to symptomatically relieve in a warm-blooded animal such as man, the symptoms of cancer, to slow the progression of cancer, or to reduce in patients with symptoms of cancer the risk of getting worse.

In this specification, unless otherwise stated, the term “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

It will be understood that compounds of formula (I) may form stable pharmaceutically acceptable acid or base salts, and in such cases administration of a compound as a salt may be appropriate. Examples of acid addition salts include acetate, adipate, ascorbate, benzoate, benzenesulfonate, bicarbonate, bisulfate, butyrate, camphorate, camphorsulfonate, choline, citrate, cyclohexyl sulfamate, diethylenediamine, ethanesulfonate, fumarate, glutamate, glycolate, hemisulfate, 2-hydroxyethylsulfonate, heptanoate, hexanoate, hydrochloride, hydrobromide, hydroiodide, hydroxymaleate, lactate, malate, maleate, methanesulfonate, meglumine, 2-naphthalenesulfonate, nitrate, oxalate, pamoate, persulfate, phenylacetate, phosphate, diphosphate, picrate, pivalate, propionate, quinate, salicylate, stearate, succinate, sulfamate, sulfanilate, sulfate, tartrate, tosylate (p-toluenesulfonate), trifluoroacetate, and undecanoate. Non-toxic physiologically-acceptable salts are preferred, although other salts may be useful, such as in isolating or purifying the product.

The salts may be formed by conventional means, such as by reacting the free base form of the product with one or more equivalents of the appropriate acid in a solvent or medium in which the salt is insoluble, or in a solvent such as water, which is removed in vacuo or by freeze drying or by exchanging the anions of an existing salt for another anion on a suitable ion-exchange resin.

Compounds of formula (I) may have more than one chiral center, and it is to be understood that the application encompasses all individual stereoisomers, enantiomers and diastereoisomers and mixtures thereof. Thus, it is to be understood that, insofar as the compounds of formula (I) can exist in optically active or racemic forms by virtue of one or more asymmetric carbon atoms, the application includes in its definition any such optically active or racemic form which possesses the above-mentioned activity. The present application encompasses all such stereoisomers having activity as herein defined.

Thus, throughout the specification, where reference is made to the compound of formula (I) it is to be understood that the term compound includes diastereoisomers, mixtures of diastereoisomers, and enantiomers that are PARP1 selective inhibitors.

It is also to be understood that certain compounds of formula (I), and pharmaceutically salts thereof, can exist in solvated as well as unsolvated forms such as, for example, hydrated and anhydrous forms. It is to be understood that the compounds herein encompass all such solvated forms. For the sake of clarity, this includes both solvated (e.g., hydrated) forms of the free form of the compound, as well as solvated (e.g., hydrated) forms of the salt of the compound.

Some of the compounds of formula (I) may be crystalline and may have more than one crystalline form. It is to be understood that the disclosure encompasses any crystalline or amorphous form, or mixtures thereof, which have PARP1 selective inhibitory activity. It is generally known that crystalline materials may be analysed using conventional techniques such as, for example, X-Ray Powder Diffraction (hereinafter XRPD) analysis and Differential Scanning calorimetry (DSC).

Formula (I) as described herein is intended to encompass all isotopes of its constituent atoms. For example, H (or hydrogen) includes any isotopic form of hydrogen including ¹H, ²H (D), and ³H (T); C includes any isotopic form of carbon including ¹²C, ¹³C, and ¹⁴C; O includes any isotopic form of oxygen including ¹⁶O, ¹⁷O and ¹⁸O; N includes any isotopic form of nitrogen including ¹³N, ¹⁴N and ¹⁵N; F includes any isotopic form of fluorine including ¹⁹F and ¹⁸F; and the like. In one aspect, the compounds of formula (I) include isotopes of the atoms covered therein in amounts corresponding to their naturally occurring abundance. However, in certain instances, it may be desirable to enrich one or more atom in a particular isotope which would normally be present in a lower abundance. For example, 1H would normally be present in greater than 99.98% abundance; however, in one aspect, a compound of any formula presented herein may be enriched in ²H or ³H at one or more positions where H is present. In another aspect, when a compound of any formula presented herein is enriched in a radioactive isotope, for example ³H and ¹⁴C, the compound may be useful in drug and/or substrate tissue distribution assays. It is to be understood that the present application encompasses all such isotopic forms.

### Description of Embodiments

Hereinafter, preferred modes for carrying out the present disclosure are described. The embodiments described below are given merely for illustrating one example of a typical embodiment of the present disclosure and are not intended to limit the scope of the present disclosure.

### 1. Antibody-Drug Conjugate

The antibody-drug conjugate used in the present disclosure is an antibody-drug conjugate in which a drug-linker represented by the following formula:

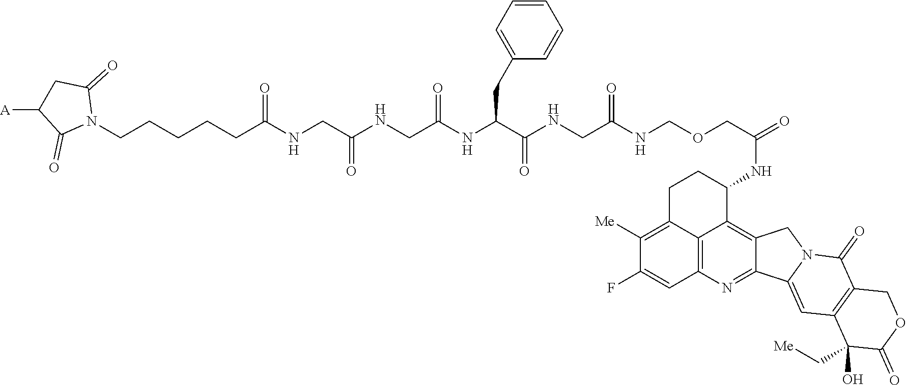

wherein A represents the connecting position to an antibody,
is conjugated to an anti-TROP2 antibody via a thioether bond.

In the present disclosure, the partial structure consisting of a linker and a drug in the antibody-drug conjugate is referred to as a “drug-linker”. The drug-linker is connected to a thiol group (in other words, the sulfur atom of a cysteine residue) formed at an interchain disulfide bond site (two sites between heavy chains, and two sites between a heavy chain and a light chain) in the antibody.

The drug-linker of the present disclosure includes exatecan (IUPAC name: (1S,9S)-1-amino-9-ethyl-5-fluoro-1,2,3,9,12,15-hexahydro-9-hydroxy-4-methyl-10H,13H-benzo[de]pyrano[3′,4′:6,7]indolizino[1,2-b]quinolin-10,13-dione, (also expressed as chemical name: (1S,9S)-1-amino-9-ethyl-5-fluoro-2,3-dihydro-9-hydroxy-4-methyl-1H, 12H-benzo[de]pyrano[3′,4′:6,7]indolizino[1,2-b]quinolin-10, 13 (9H,15H)-dione)), which is a topoisomerase I inhibitor, as a component. Exatecan is a camptothecin derivative having an antitumor effect, represented by the following formula:

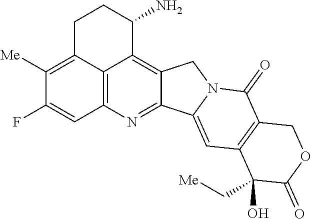 [SMILES: CC[C@@]1(O)C(=O)OCc2c1cc1n(c2=O)Cc2c-1nc1cc(F)c(C)c3c1c2[C@@H](N)CC3]

The anti-TROP2 antibody-drug conjugate used in the present disclosure can be also represented by the following formula:

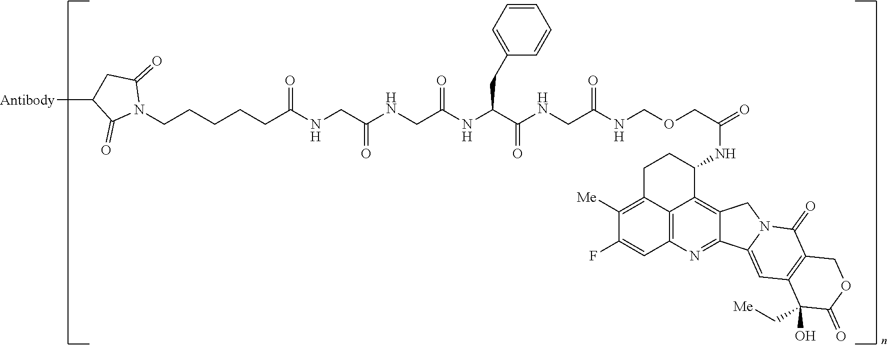

Here, the drug-linker is conjugated to an anti-TROP2 antibody (‘Antibody-’) via a thioether bond. The meaning of n is the same as that of what is called the average number of conjugated drug molecules (DAR; Drug-to-Antibody Ratio), and indicates the average number of units of the drug-linker conjugated per antibody molecule.

After migrating into cancer cells, the anti-TROP2 antibody-drug conjugate used in the present disclosure is cleaved at the linker portion to release a compound represented by the following formula:

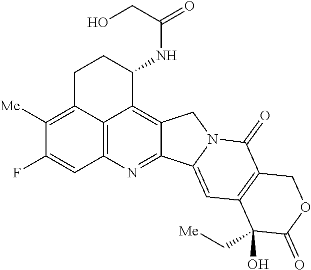 [SMILES: CC[C@@]1(O)C(=O)OCc2c1cc1n(c2=O)Cc2c-1nc1cc(F)c(C)c3c1c2[C@@H](NC(=O)CO)CC3]

### 2. Anti-TROP2 Antibody in Antibody-Drug Conjugate

The anti-TROP2 antibody in the antibody-drug conjugate used in the present invention may be derived from any species and is preferably an antibody derived from a human, a rat, a mouse, or a rabbit. In cases when the antibody is derived from species other than human species, it is preferably chimerized or humanized using a well known technique. The antibody of the present invention may be a polyclonal antibody or a monoclonal antibody and is preferably a monoclonal antibody.

The antibody in the antibody-drug conjugate used in the present invention is an antibody preferably having the characteristic of being able to target cancer cells, and is preferably an antibody possessing, for example, the property of being able to recognize a cancer cell, the property of being able to bind to a cancer cell, the property of being internalized in a cancer cell, and/or cytocidal activity against cancer cells.

The binding activity of the antibody against cancer cells can be confirmed using flow cytometry. The internalization of the antibody into tumor cells can be confirmed using (1) an assay of visualizing an antibody incorporated in cells under a fluorescence microscope using a secondary antibody (fluorescently labeled) binding to the therapeutic antibody (Cell Death and Differentiation (2008) 15, 751-761), (2) an assay of measuring a fluorescence intensity incorporated in cells using a secondary antibody (fluorescently labeled) binding to the therapeutic antibody (Molecular Biology of the Cell, Vol. 15, 5268-5282 December 2004), or (3) a Mab-ZAP assay using an immunotoxin binding to the therapeutic antibody wherein the toxin is released upon incorporation into cells to inhibit cell growth (Bio Techniques 28:162-165, January 2000). As the immunotoxin, a recombinant complex protein of a diphtheria toxin catalytic domain and protein G may be used.

The antitumor activity of the antibody can be confirmed in vitro by determining inhibitory activity against cell growth. For example, a cancer cell line overexpressing a target protein for the antibody is cultured, and the antibody is added at varying concentrations into the culture system to determine inhibitory activity against focus formation, colony formation, and spheroid growth. The antitumor activity can be confirmed in vivo, for example, by administering the antibody to a nude mouse with a transplanted cancer cell line highly expressing the target protein, and determining changes in the cancer cells.

Since the compound conjugated in the antibody-drug conjugate exerts an antitumor effect, it is preferred but not essential that the antibody itself should have an antitumor effect. For the purpose of specifically and selectively exerting the cytotoxic activity of the antitumor compound against cancer cells, it is important and also preferred that the antibody should have the property of being internalized to migrate into cancer cells.

The antibody in the antibody-drug conjugate used in the present invention can be obtained by a procedure known in the art. For example, the antibody of the present invention can be obtained using a method usually carried out in the art, which involves immunizing animals with an antigenic polypeptide and collecting and purifying antibodies produced in vivo. The origin of the antigen is not limited to humans, and the animals may be immunized with an antigen derived from a non-human animal such as a mouse, a rat and the like. In this case, the cross-reactivity of antibodies binding to the obtained heterologous antigen with human antigens can be tested to screen for an antibody applicable to a human disease.

Alternatively, antibody-producing cells which produce antibodies against the antigen can be fused with myeloma cells according to a method known in the art (for example, Kohler and Milstein, Nature (1975) 256, p. 495-497; Kennet, R. ed., Monoclonal Antibodies, p. 365-367, Plenum Press, N.Y. (1980)), to establish hybridomas, from which monoclonal antibodies can in turn be obtained.

The antigen can be obtained by genetically engineering host cells to produce a gene encoding the antigenic protein. Specifically, vectors that permit expression of the antigen gene are prepared and transferred to host cells so that the gene is expressed. The antigen thus expressed can be purified. The antibody can also be obtained by a method of immunizing animals with the above-described genetically engineered antigen-expressing cells or a cell line expressing the antigen.

The antibody in the antibody-drug conjugate used in the present invention is preferably a recombinant antibody obtained by artificial modification for the purpose of decreasing heterologous antigenicity to humans such as a chimeric antibody or a humanized antibody, or is preferably an antibody having only the gene sequence of an antibody derived from a human, that is, a human antibody. These antibodies can be produced using a known method.

As the chimeric antibody, an antibody in which antibody variable and constant regions are derived from different species, for example, a chimeric antibody in which a mouse- or rat-derived antibody variable region is connected to a human-derived antibody constant region can be exemplified (Proc. Natl. Acad. Sci. USA, 81, 6851-6855, (1984)).

As the humanized antibody, an antibody obtained by integrating only the complementarity determining region (CDR) of a heterologous antibody into a human-derived antibody (Nature (1986) 321, pp. 522-525), an antibody obtained by grafting a part of the amino acid residues of the framework of a heterologous antibody as well as the CDR sequence of the heterologous antibody to a human antibody by a CDR-grafting method (WO 90/07861), and an antibody humanized using a gene conversion mutagenesis strategy (U.S. Pat. No. 5,821,337) can be exemplified.

As the human antibody, an antibody generated by using a human antibody-producing mouse having a human chromosome fragment including genes of a heavy chain and light chain of a human antibody (see Tomizuka, K. et al., Nature Genetics (1997) 16, p. 133-143; Kuroiwa, Y. et. al., Nucl. Acids Res. (1998) 26, p. 3447-3448; Yoshida, H. et. al., Animal Cell Technology: Basic and Applied Aspects vol. 10, p. 69-73 (Kitagawa, Y., Matsuda, T. and Iijima, S. eds.), Kluwer Academic Publishers, 1999; Tomizuka, K. et. al., Proc. Natl. Acad. Sci. USA (2000) 97, p. 722-727, etc.) can be exemplified. As an alternative, an antibody obtained by phage display, the antibody being selected from a human antibody library (see Wormstone, I. M. et. al, Investigative Ophthalmology & Visual Science. (2002) 43 (7), p. 2301-2308; Carmen, S. et. al., Briefings in Functional Genomics and Proteomics (2002), 1 (2), p. 189-203; Siriwardena, D. et. al., Ophthalmology (2002) 109 (3), p. 427-431, etc.) can be exemplified.

In the antibody in the antibody-drug conjugate used in present invention, modified variants of the antibody are also included. The modified variant refers to a variant obtained by subjecting the antibody according to the present invention to chemical or biological modification. Examples of the chemically modified variant include variants including a linkage of a chemical moiety to an amino acid skeleton, variants including a linkage of a chemical moiety to an N-linked or O-linked carbohydrate chain, etc. Examples of the biologically modified variant include variants obtained by post-translational modification (such as N-linked or O-linked glycosylation, N- or C-terminal processing, deamidation, isomerization of aspartic acid, or oxidation of methionine), and variants in which a methionine residue has been added to the N terminus by being expressed in a prokaryotic host cell. Further, an antibody labeled so as to enable the detection or isolation of the antibody or an antigen according to the present invention, for example, an enzyme-labeled antibody, a fluorescence-labeled antibody, and an affinity-labeled antibody are also included in the meaning of the modified variant. Such a modified variant of the antibody according to the present invention is useful for improving the stability and blood retention of the antibody, reducing the antigenicity thereof, detecting or isolating an antibody or an antigen, and so on.

Further, by regulating the modification of a glycan which is linked to the antibody according to the present invention (glycosylation, defucosylation, etc.), it is possible to enhance antibody-dependent cellular cytotoxic activity. As the technique for regulating the modification of a glycan of antibodies, International Publication No. WO 99/54342, International Publication No. WO 00/61739, International Publication No. WO 02/31140, International Publication No. WO 2007/133855, International Publication No. WO 2013/120066, etc. are known. However, the technique is not limited thereto. In the antibody according to the present invention, antibodies in which the modification of a glycan is regulated are also included.

It is known that a lysine residue at the carboxyl terminus of the heavy chain of an antibody produced in a cultured mammalian cell is deleted (Journal of Chromatography A, 705:129-134 (1995)), and it is also known that two amino acid residues (glycine and lysine) at the carboxyl terminus of the heavy chain of an antibody produced in a cultured mammalian cell are deleted and a proline residue newly located at the carboxyl terminus is amidated (Analytical Biochemistry, 360:75-83 (2007)). However, such deletion and modification of the heavy chain sequence do not affect the antigen-binding affinity and the effector function (complement activation, antibody-dependent cellular cytotoxicity, etc.) of the antibody. Therefore, in the antibody according to the present invention, antibodies subjected to such modification and functional fragments of the antibody are also included, and deletion variants in which one or two amino acids have been deleted at the carboxyl terminus of the heavy chain, variants obtained by amidation of the deletion variants (for example, a heavy chain in which the carboxyl terminal proline residue has been amidated), and the like are also included. The type of deletion variant having a deletion at the carboxyl terminus of the heavy chain of the antibody according to the present invention is not limited to the above variants as long as the antigen-binding affinity and the effector function are conserved. The two heavy chains constituting the antibody according to the present invention may be of one type selected from the group consisting of a full-length heavy chain and the above-described deletion variant, or may be of two types in combination selected therefrom. The ratio of the amount of each deletion variant can be affected by the type of cultured mammalian cells which produce the antibody according to the present invention and the culture conditions; however, an antibody in which one amino acid residue at the carboxyl terminus has been deleted in both of the two heavy chains in the antibody according to the present invention can be preferably exemplified.

As isotypes of the antibody according to the present invention, for example, IgG (IgG1, IgG2, IgG3, IgG4) can be exemplified. Preferably, IgG1 or IgG2 can be exemplified.

In the present invention, the term “anti-TROP2 antibody” refers to an antibody which binds specifically to TROP2 (TACSTD2: Tumor-associated calcium signal transducer 2; EGP-1), and preferably has an activity of internalization in TROP2-expressing cells by binding to TROP2.

Examples of the anti-TROP2 antibody include hTINA1-H1L1 (WO 2015/098099).

### 3. Production of Antibody-Drug Conjugate

A drug-linker intermediate for use in the production of the antibody-drug conjugate according to the present invention is represented by the following formula.

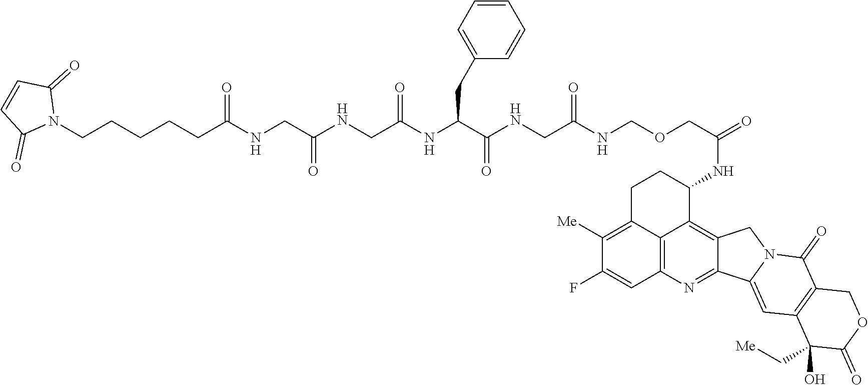 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)[C@H](Cc1ccccc1)NC(=O)CNC(=O)CNC(=O)CCCCCN1C(=O)C=CC1=O)CC3]

The drug-linker intermediate can be expressed as the chemical name N-[6-(2,5-dioxo-2,5-dihydro-1H-pyrrol-1-yl) hexanoyl]glycylglycyl-L-phenylalanyl-N-[(2-{[(1,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl]amino}-2-oxoethoxy)methyl]glycinamide, and can be produced with reference to descriptions in WO 2014/057687, WO 2015/098099, WO 2015/115091, WO 2015/155998, WO 2019/044947, and so on.

The antibody-drug conjugate used in the present invention can be produced by reacting the above-described drug-linker intermediate and an anti-TROP2 antibody having a thiol group (alternatively referred to as a sulfhydryl group).

The anti-TROP2 antibody having a sulfhydryl group can be obtained by a method well known in the art (Hermanson, G. T, Bioconjugate Techniques, pp. 56-136, pp. 456-493, Academic Press (1996)). For example, by using 0.3 to 3 molar equivalents of a reducing agent such as tris(2-carboxyethyl) phosphine hydrochloride (TCEP) per interchain disulfide within the antibody and reacting with the antibody in a buffer solution containing a chelating agent such as ethylenediamine tetraacetic acid (EDTA), an antibody having a sulfhydryl group with partially or completely reduced interchain disulfides within the antibody can be obtained.

Further, by using 2 to 20 molar equivalents of the drug-linker intermediate per the antibody having a sulfhydryl group, an antibody-drug conjugate in which 2 to 8 drug molecules are conjugated per antibody molecule can be produced.

The average number of conjugated drug molecules per antibody molecule of the antibody-drug conjugate produced can be determined, for example, by a method of calculation based on measurement of UV absorbance for the antibody-drug conjugate and the conjugation precursor thereof at two wavelengths of 280 nm and 370 nm (UV method), or a method of calculation based on quantification through HPLC measurement for fragments obtained by treating the antibody-drug conjugate with a reducing agent (HPLC method).

Conjugation between the antibody and the drug-linker intermediate and calculation of the average number of conjugated drug molecules per antibody molecule of the antibody-drug conjugate can be performed with reference to descriptions in WO 2015/098099 and WO 2017/002776, for example.

In the present invention, the term “anti-TROP2 antibody-drug conjugate” refers to an antibody-drug conjugate such that the antibody in the antibody-drug conjugate according to the invention is an anti-TROP2 antibody.

The anti-TROP2 antibody is preferably an antibody comprising a heavy chain comprising CDRH1 consisting of an amino acid sequence represented by SEQ ID NO: 3 [=an amino acid sequence consisting of amino acid residues 50 to 54 of SEQ ID NO: 1], CDRH2 consisting of an amino acid sequence represented by SEQ ID NO: 4 [=an amino acid sequence consisting of amino acid residues 69 to 85 of SEQ ID NO: 1], and CDRH3 consisting of an amino acid sequence represented by SEQ ID NO: 5 [=an amino acid sequence consisting of amino acid residues 118 to 129 of SEQ ID NO: 1], and a light chain comprising CDRL1 consisting of an amino acid sequence represented by SEQ ID NO: 6 [=an amino acid sequence consisting of amino acid residues 44 to 54 of SEQ ID NO: 2], CDRL2 consisting of an amino acid sequence represented by SEQ ID NO: 7 [=an amino acid sequence consisting of amino acid residues 70 to 76 of SEQ ID NO: 2], and CDRL3 consisting of an amino acid sequence represented by SEQ ID NO: 8 [=an amino acid sequence consisting of amino acid residues 109 to 117 of SEQ ID NO: 2],
more preferably an antibody comprising a heavy chain comprising a heavy chain variable region consisting of an amino acid sequence represented by SEQ ID NO: 9 [=an amino acid sequence consisting of amino acid residues 20 to 140 of SEQ ID NO: 1], and a light chain comprising a light chain variable region consisting of an amino acid sequence represented by SEQ ID NO: 10 [=an amino acid sequence consisting of amino acid residues 21 to 129 of SEQ ID NO: 2], and
even more preferably an antibody comprising a heavy chain consisting of an amino acid sequence represented by SEQ ID NO: 12 [=an amino acid sequence consisting of amino acid residues 20 to 470 of SEQ ID NO: 1] and a light chain consisting of an amino acid sequence represented by SEQ ID NO: 13 [=amino acid residues 21 to 234 of SEQ ID NO: 2], or an antibody comprising a heavy chain consisting of an amino acid sequence represented by SEQ ID NO: 11 [=an amino acid sequence consisting of amino acid residues 20 to 469 of SEQ ID NO: 1] and a light chain consisting of an amino acid sequence represented by SEQ ID NO: 13 [=amino acid residues 21 to 234 of SEQ ID NO: 2].

The average number of units of the drug-linker conjugated per antibody molecule in the anti-TROP2 antibody-drug conjugate is preferably 2 to 8, more preferably 3 to 5, even more preferably 3.5 to 4.5, and even more preferably about 4.

The anti-TROP2 antibody-drug conjugate can be produced with reference to descriptions in WO 2015/098099 and WO 2017/002776.

In preferred embodiments, the anti-TROP2 antibody-drug conjugate is datopotamab deruxtecan (DS-1062).

### 4. PARP1 Selective Inhibitor

In the present disclosure, the term “PARP1 selective inhibitor” refers to a PARP inhibitor that exhibits selectivity for PARP1 over other PARP family members such as PARP2, PARP3, PARP5a, and PARP6, advantageously selectivity for PARP1 over PARP2, preferably at least 10-fold selectivity for PARP1 over PARP2, and more preferably at least 100-fold selectivity for PARP1 over PARP2. Preferred examples of PARP1 selective inhibitors can include those disclosed herein.

Examples of PARP1 selective inhibitors which may be used according to the present disclosure include azaquinolone compounds of formula (I). Azaquinolone compounds of formula (I) described herein have surprisingly high selectivity for PARP1 over other PARP family members such as PARP2, PARP3, PARP5a, and PARP6. Advantageously, compounds of formula (I) described herein have low hERG activity. It is well known that blockade of the cardiac ion channel coded by human ether-à-gogo-related gene (hERG) is a risk factor in drug discovery and development, and that blockage of hERG can cause safety problems such as cardiac arrhythmia.

Accordingly, in preferred embodiments of the PARP1 selective inhibitor used in the present disclosure, the PARP1 selective inhibitor is a compound represented by the following formula (I):

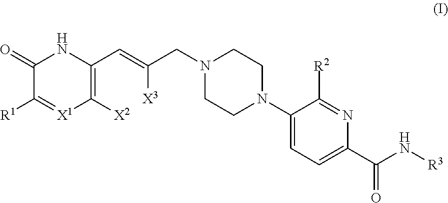

wherein:
X¹ and X² are each independently selected from N and C(H),
X³ is independently selected from N and C(R⁴), wherein R⁴ is H or fluoro,
R¹ is C₁₋₄ alkyl or C₁₋₄ fluoroalkyl (preferably is ethyl),
R² is independently selected from H, halo, C₁₋₄ alkyl, and C₁₋₄ fluoroalkyl, and
R³ is H or C₁₋₄ alkyl (preferably is C₁₋₄ alkyl, more preferably methyl),
or a pharmaceutically acceptable salt thereof
provided that:
when X¹ is N, then X² is C(H), and X³ is C(R⁴),
when X² is N, then X¹═C(H), and X³ is C(R⁴), and
when X³ is N, then X¹ and X² are both C(H).

In one embodiment the PARP1 selective inhibitor used in the disclosure is a compound of formula (Ia):

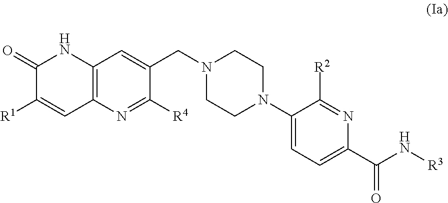

wherein
R¹ is C₁₋₄ alkyl, R² is selected from H, halo, C₁₋₄ alkyl, and C₁₋₄ fluoroalkyl (preferably is selected from difluoromethyl, trifluoromethyl, and methyl, or is H or halo), R³ is H or C₁₋₄ alkyl, and R⁴ is H. In the compound of formula (Ia), preferably R¹ is ethyl, R² is selected from H, chloro and fluoro, R³ is methyl, and R⁴ is H.

In another embodiment the PARP1 selective inhibitor used in the disclosure is a compound of formula (Ib):

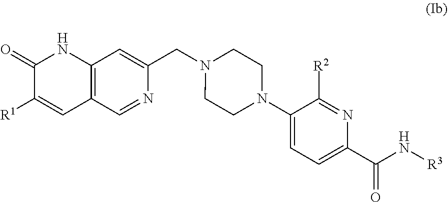

wherein
R¹ is C₁₋₄ alkyl, R² is H or halo, and R³ is H or C₁₋₄ alkyl. In the compound of formula (Ib), preferably R¹ is ethyl, R² is selected from H, chloro and fluoro, and R³ is methyl.

In another embodiment the PARP1 selective inhibitor used in the disclosure is a compound of formula (Ic):

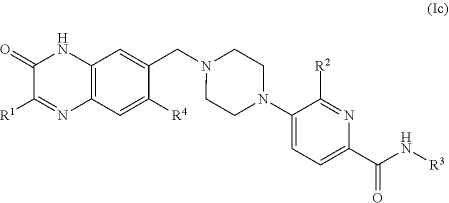

wherein
R¹ is C₁₋₄ alkyl or C₁₋₄ fluoroalkyl, R² is independently selected from H, halo, C₁₋₄ alkyl, and C₁₋₄ fluoroalkyl, R³ is H or C₁₋₄ alkyl, and R⁴ is H or fluoro.

In another embodiment the PARP1 selective inhibitor is a compound of formula (Ic) wherein:
R¹ is independently selected from ethyl, n-propyl, trifluoromethyl, 1,1-difluoroethyl, 2,2-difluroethyl, 2-fluoroethyl, and 2,2,2-trifluoroethyl; R² is independently selected from H, methyl, ethyl, trifluoromethyl, difluoromethyl, fluoromethyl, fluoro, and chloro; R³ is H or methyl, and R⁴ is H.

In another embodiment the PARP1 selective inhibitor is a compound of formula (I), or of formula (Ia), (Ib) or (Ic), having selectivity for PARP1 over PARP2, preferably at least 10-fold selectivity for PARP1 over PARP2, and more preferably at least 100-fold selectivity for PARP1 over PARP2.

In other embodiments the PARP1 selective inhibitor used in the disclosure is a compound selected from:
5-[4-[(3-ethyl-2-oxo-1H-1,6-naphthyridin-7-yl)methyl]piperazin-1-yl]-N-methyl-pyridine-2-carboxamide,
5-[4-[(3-ethyl-2-oxo-1H-1,6-naphthyridin-7-yl)methyl]piperazin-1-yl]-6-fluoro-N-methyl-pyridine-2-carboxamide,
6-chloro-5-[4-[(3-ethyl-2-oxo-1H-1,6-naphthyridin-7-yl)methyl]piperazin-1-yl]-N-methyl-pyridine-2-carboxamide,
5-[4-[(7-ethyl-6-oxo-5H-1,5-naphthyridin-3-yl)methyl]piperazin-1-yl]-N-methyl-pyridine-2-carboxamide,
5-[4-[(7-ethyl-6-oxo-5H-1,5-naphthyridin-3-yl)methyl]piperazin-1-yl]-6-fluoro-N-methyl-pyridine-2-carboxamide,
6-chloro-5-[4-[(7-ethyl-6-oxo-5H-1,5-naphthyridin-3-yl)methyl]piperazin-1-yl]-N-methyl-pyridine-2-carboxamide,
5-[4-[(7-ethyl-6-oxo-5H-1,5-naphthyridin-3-yl)methyl]piperazin-1-yl]pyridine-2-carboxamide
6-ethyl-5-[4-[(2-ethyl-3-oxo-4H-quinoxalin-6-yl)methyl]piperazin-1-yl]-N-methyl-pyridine-2-carboxamide,
5-[4-[(2-ethyl-3-oxo-4H-quinoxalin-6-yl)methyl]piperazin-1-yl]-N-methyl-6-(trifluoromethyl)pyridine-2-carboxamide,
6-(difluoromethyl)-5-[4-[(2-ethyl-3-oxo-4H-quinoxalin-6-yl)methyl]piperazin-1-yl]-N-methyl-pyridine-2-carboxamide,
5-[4-[(2-ethyl-3-oxo-4H-quinoxalin-6-yl)methyl]piperazin-1-yl]-N-methyl-pyridine-2-carboxamide,
5-[4-[(2-ethyl-3-oxo-4H-quinoxalin-6-yl)methyl]piperazin-1-yl]-6-fluoro-N-methyl-pyridine-2-carboxamide,
5-[4-[(2-ethyl-3-oxo-4H-quinoxalin-6-yl)methyl]piperazin-1-yl]-N, 6-dimethyl-pyridine-2-carboxamide,
6-chloro-5-[4-[(2-ethyl-3-oxo-4H-quinoxalin-6-yl)methyl]piperazin-1-yl]-N-methyl-pyridine-2-carboxamide,
N-methyl-5-[4-[[3-oxo-2-(trifluoromethyl)-4H-quinoxalin-6-yl]methyl]piperazin-1-yl]pyridine-2-carboxamide,
6-chloro-N-methyl-5-[4-[[3-oxo-2-(trifluoromethyl)-4H-quinoxalin-6-yl]methyl]piperazin-1-yl]pyridine-2-carboxamide,
6-fluoro-N-methyl-5-[4-[[3-oxo-2-(trifluoromethyl)-4H-quinoxalin-6-yl]methyl]piperazin-1-yl]pyridine-2-carboxamide,
N-methyl-5-[4-[(3-oxo-2-propyl-4H-quinoxalin-6-yl)methyl]piperazin-1-yl]pyridine-2-carboxamide,
6-chloro-N-methyl-5-[4-[(3-oxo-2-propyl-4H-quinoxalin-6-yl)methyl]piperazin-1-yl]pyridine-2-carboxamide,
6-fluoro-N-methyl-5-[4-[(3-oxo-2-propyl-4H-quinoxalin-6-yl)methyl]piperazin-1-yl]pyridine-2-carboxamide,
5-[4-[(2-ethyl-7-fluoro-3-oxo-4H-quinoxalin-6-yl)methyl]piperazin-1-yl]-6-fluoro-N-methyl-pyridine-2-carboxamide,
5-[4-[[2-(1,1-difluoroethyl)-3-oxo-4H-quinoxalin-6-yl]methyl]piperazin-1-yl]-N-methyl-pyridine-2-carboxamide,
5-[4-[[2-(2,2-difluoroethyl)-3-oxo-4H-quinoxalin-6-yl]methyl]piperazin-1-yl]-N-methyl-pyridine-2-carboxamide,
5-[4-[[2-(2,2-difluoroethyl)-3-oxo-4H-quinoxalin-6-yl]methyl]piperazin-1-yl]-6-fluoro-N-methyl-pyridine-2-carboxamide,
5-[4-[[2-(2-fluoroethyl)-3-oxo-4H-quinoxalin-6-yl]methyl]piperazin-1-yl]-N-methyl-pyridine-2-carboxamide,
6-fluoro-5-[4-[[2-(2-fluoroethyl)-3-oxo-4H-quinoxalin-6-yl]methyl]piperazin-1-yl]-N-methyl-pyridine-2-carboxamide,
N-methyl-5-[4-[[3-oxo-2-(2,2,2-trifluoroethyl)-4H-quinoxalin-6-yl]methyl]piperazin-1-yl]pyridine-2-carboxamide, and
6-fluoro-N-methyl-5-(4-((3-oxo-2-(2,2,2-trifluoroethyl)-3,4-dihydroquinoxalin-6-yl)methyl) piperazin-1-yl) picolinamide,
or a pharmaceutically acceptable salt thereof

In another embodiment the PARP1 selective inhibitor used in the disclosure is a compound selected from:
6-(difluoromethyl)-5-[4-[(7-ethyl-6-oxo-5H-1,5-naphthyridin-3-yl)methyl]piperazin-1-yl]-N-methyl-pyridine-2-carboxamide,
5-[4-[(7-ethyl-6-oxo-5H-1,5-naphthyridin-3-yl)methyl]piperazin-1-yl]-N-methyl-6 (trifluoromethyl)pyridine-2-carboxamide,
5-[4-[(7-ethyl-6-oxo-5H-1,5-naphthyridin-3-yl)methyl]piperazin-1-yl]-N, 6-dimethyl-pyridine-2-carboxamide, and
N-ethyl-5-[4-[(7-ethyl-6-oxo-5H-1,5-naphthyridin-3-yl)methyl]piperazin-1-yl]pyridine-2-carboxamide,
or a pharmaceutically acceptable salt thereof.

In a preferred embodiment the PARP1 selective inhibitor used in the disclosure is the compound AZD5305 (5-[4-[(7-ethyl-6-oxo-5H-1,5-naphthyridin-3-yl)methyl]piperazin-1-yl]-N-methyl-pyridine-2-carboxamide) represented by the following formula:

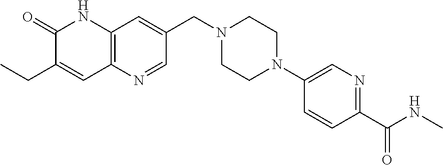 [SMILES: CCc1cc2ncc(CN3CCN(c4ccc(C(=O)NC)nc4)CC3)cc2[nH]c1=O]

or a pharmaceutically acceptable salt thereof.

### 5. Combination of Antibody-Drug Conjugate and PARP1 Selective Inhibitor

In a first combination embodiment of the disclosure, the anti-TROP2 antibody-drug conjugate which is combined with the PARP1 selective inhibitor is an antibody-drug conjugate in which a drug-linker represented by the following formula:

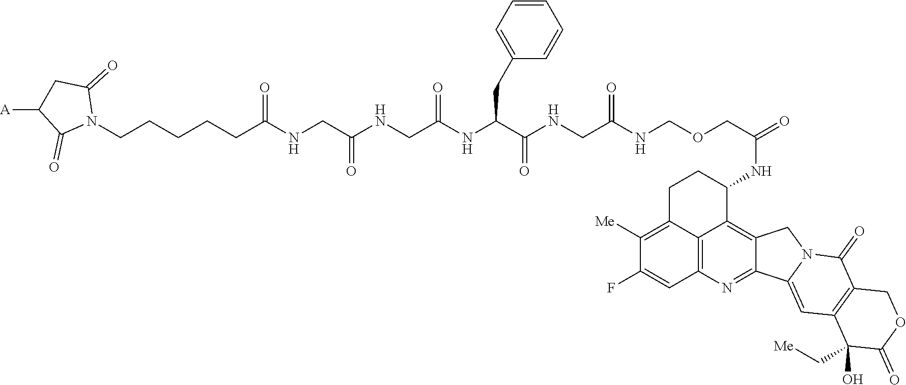

wherein A represents the connecting position to an antibody, is conjugated to an anti-TROP2 antibody via a thioether bond.

In another combination embodiment, the anti-TROP2 antibody-drug conjugate as defined above for the first combination embodiment is combined with a PARP1 selective inhibitor which is a compound represented by the following formula (I):

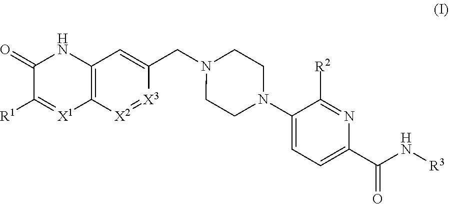

wherein:
X¹ and X² are each independently selected from N and C(H),
X³ is independently selected from N and C(R⁴), wherein R⁴ is H or fluoro,
R¹ is C₁₋₄ alkyl or C₁₋₄ fluoroalkyl,
R² is independently selected from H, halo, C₁₋₄ alkyl, and
C₁₋₄ fluoroalkyl, and
R³ is H or C₁₋₄ alkyl,
or a pharmaceutically acceptable salt thereof
provided that:
when X¹ is N, then X² is C(H), and X³ is C(R⁴),
when X² is N, then X¹═C(H), and X³ is C(R⁴), and
when X³ is N, then X¹ and X² are both C(H).

In another combination embodiment, the anti-TROP2 antibody-drug conjugate as defined above is combined with a PARP1 selective inhibitor as defined above wherein, in formula (I), R³ is C₁₋₄ alkyl.

In another combination embodiment, the anti-TROP2 antibody-drug conjugate as defined above is combined with a PARP1 selective inhibitor as defined above wherein, in formula (I), R³ is methyl.

In another combination embodiment, the anti-TROP2 antibody-drug conjugate as defined above is combined with a PARP1 selective inhibitor as defined above wherein, in formula (I), R¹ is ethyl.

In another combination embodiment, the anti-TROP2 antibody-drug conjugate as defined above is combined with a PARP1 selective inhibitor which is a compound represented by the following formula (Ia):

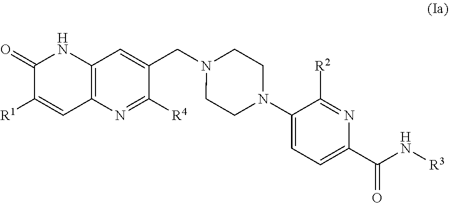

wherein
R¹ is C₁₋₄ alkyl,
R² is selected from H, halo, C₁₋₄ alkyl, and C₁₋₄ fluoroalkyl,
R³ is H or C₁₋₄ alkyl, and
R⁴ is H,
or a pharmaceutically acceptable salt thereof.

In another combination embodiment, the anti-TROP2 antibody-drug conjugate as defined above is combined with a PARP1 selective inhibitor as defined above wherein, in formula (Ia), R² is H or halo.

In another combination embodiment, the anti-TROP2 antibody-drug conjugate as defined above is combined with a PARP1 selective inhibitor as defined above wherein, in formula (Ia), R¹ is ethyl, R² is selected from H, chloro and fluoro, and R³ is methyl.

In another combination embodiment, the anti-TROP2 antibody-drug conjugate as defined above is combined with a PARP1 selective inhibitor wherein the PARP1 selective inhibitor is AZD5305 represented by the following formula:

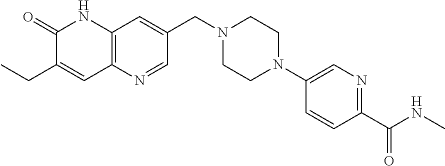 [SMILES: CCc1cc2ncc(CN3CCN(c4ccc(C(=O)NC)nc4)CC3)cc2[nH]c1=O]

or a pharmaceutically acceptable salt thereof.

In an embodiment of each of the combination embodiments described above, the anti-TROP2 antibody comprises a heavy chain comprising CDRH1 consisting of an amino acid sequence represented by SEQ ID NO: 3 [=amino acid residues 50 to 54 of SEQ ID NO: 1], CDRH2 consisting of an amino acid sequence represented by SEQ ID NO: 4 [=amino acid residues 69 to 85 of SEQ ID NO: 1] and CDRH3 consisting of an amino acid sequence represented by SEQ ID NO: 5 [=amino acid residues 118 to 129 of SEQ ID NO: 1], and a light chain comprising CDRL1 consisting of an amino acid sequence represented by SEQ ID NO: 6 [=amino acid residues 44 to 54 of SEQ ID NO: 2], CDRL2 consisting of an amino acid sequence represented by SEQ ID NO: 7 [=amino acid residues 70 to 76 of SEQ ID NO: 2] and CDRL3 consisting of an amino acid sequence represented by SEQ ID NO: 8 [=amino acid residues 109 to 117 of SEQ ID NO: 2]. In another embodiment of each of the combination embodiments described above, the anti-TROP2 antibody comprises a heavy chain comprising a heavy chain variable region consisting of an amino acid sequence represented by SEQ ID NO: 9 [=amino acid residues 20 to 140 of SEQ ID NO: 1] and a light chain comprising a light chain variable region consisting of an amino acid sequence represented by SEQ ID NO: 10 [=amino acid residues 21 to 129 of SEQ ID NO: 2]. In another embodiment of each of the combination embodiments described above, the anti-TROP2 antibody comprises a heavy chain consisting of an amino acid sequence represented by SEQ ID NO: 12 [=amino acid residues 20 to 470 of SEQ ID NO: 1] and a light chain consisting of an amino acid sequence represented by SEQ ID NO: 13 [=amino acid residues 21 to 234 of SEQ ID NO: 2]. In another embodiment of each of the combination embodiments described above, the anti-TROP2 antibody comprises a heavy chain consisting of an amino acid sequence represented by SEQ ID NO: 11 [=amino acid residues 20 to 469 of SEQ ID NO: 1] and a light chain consisting of an amino acid sequence represented by SEQ ID NO: 13 [=amino acid residues 21 to 234 of SEQ ID NO: 2].

In a particularly preferred combination embodiment of the disclosure, the anti-TROP2 antibody-drug conjugate is datopotamab deruxtecan (DS-1062) and the PARP1 selective inhibitor is the compound represented by the following formula:

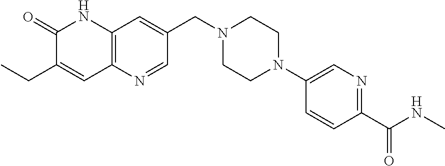 [SMILES: CCc1cc2ncc(CN3CCN(c4ccc(C(=O)NC)nc4)CC3)cc2[nH]c1=O]

also identified as AZD5305.

### 6. Therapeutic Combined Use and Method

Described in the following are a pharmaceutical product and a therapeutic use and method wherein the anti-TROP2 antibody-drug conjugate according to the present disclosure and a PARP1 selective inhibitor are administered in combination.

The pharmaceutical product and therapeutic use and method of the present disclosure may be characterized in that the anti-TROP2 antibody-drug conjugate and the PARP1 selective inhibitor are separately contained as active components in different formulations, and are administered simultaneously or at different times, or characterized in that the antibody-drug conjugate and the PARP1 selective inhibitor are contained as active components in a single formulation and administered.

In the pharmaceutical product and therapeutic method of the present disclosure, a single PARP1 selective inhibitor used in the present disclosure can be administered in combination with the anti-TROP2 antibody-drug conjugate, or two or more different PARP1 selective inhibitors can be administered in combination with the antibody-drug conjugate.

The pharmaceutical product and therapeutic method of the present disclosure can be used for treating cancer, and can be preferably used for treating at least one cancer selected from the group consisting of breast cancer (including triple negative breast cancer and hormone receptor (HR)-positive, HER2-negative breast cancer), lung cancer (including small cell lung cancer and non-small cell lung cancer), colorectal cancer (also called colon and rectal cancer, and including colon cancer and rectal cancer), gastric cancer (also called gastric adenocarcinoma), esophageal cancer, head-and-neck cancer (including salivary gland cancer and pharyngeal cancer), esophagogastric junction adenocarcinoma, biliary tract cancer (including bile duct cancer), Paget's disease, pancreatic cancer, ovarian cancer, uterine carcinosarcoma, urothelial cancer, prostate cancer, bladder cancer, gastrointestinal stromal tumor, cervical cancer (including uterine cervix cancer), squamous cell carcinoma, peritoneal cancer, liver cancer, hepatocellular cancer, corpus uteri carcinoma, kidney cancer, vulval cancer, thyroid cancer, penis cancer, leukemia, malignant lymphoma, plasmacytoma, myeloma, glioblastoma multiforme, osteosarcoma, sarcoma, endometrial cancer, and melanoma, and can be more preferably used for treating at least one cancer selected from the group consisting of breast cancer (preferably triple negative breast cancer and hormone receptor (HR)-positive, HER2-negative breast cancer), lung cancer (preferably non-small cell lung cancer, including non-small cell lung cancer with actionable genomic alterations and non-small cell lung cancer without actionable genomic alterations, wherein the actionable genomic alterations include EGFR, ALK, ROS1, NTRK, BRAF, RET, and MET exon 14 skipping), colorectal cancer, gastric cancer, pancreatic cancer, ovarian cancer, prostate cancer, kidney cancer, and endometrial cancer. Furthermore, the pharmaceutical product and therapeutic method of the present disclosure can preferably be used for treating cancer that is deficient in Homologous Recombination (HR) dependent DNA DSB repair activity, or cancer that is not deficient in Homologous Recombination (HR) dependent DNA DSB repair activity. Furthermore, the pharmaceutical product and therapeutic method of the present disclosure can preferably be used for treating cancer that exhibits resistance or refractoriness to a previous treatment with a PARP inhibitor (in particular a PARP inhibitor selected from olaparib, rucaparib, niraparib, talazoparib and veliparib). The presence or absence of TROP2 tumor markers can be determined, for example, by collecting tumor tissue from a cancer patient to prepare a formalin-fixed, paraffin-embedded (FFPE) specimen and subjecting the specimen to a test for gene products (proteins), for example, with an immunohistochemical (IHC) method, a flow cytometer, or Western blotting, or to a test for gene transcription, for example, with an in situ hybridization (ISH) method, a quantitative PCR method (q-PCR), or microarray analysis, or by collecting cell-free circulating tumor DNA (ctDNA) from a cancer patient and subjecting the ctDNA to a test with a method such as next-generation sequencing (NGS).

The pharmaceutical product and therapeutic method of the present disclosure can be preferably used for mammals, and can be more preferably used for humans.

The antitumor effect of the pharmaceutical product and therapeutic method of the present disclosure can be confirmed by, for example, generating a model in which cancer cells are transplanted to a test animal, and measuring reduction in tumor volume, life-prolonging effects due to applying the pharmaceutical product and therapeutic method of the present disclosure. Furthermore, comparison with the antitumor effect of single administration of each of the antibody-drug conjugate and the PARP1 selective inhibitor used in the present invention can provide confirmation of the combined effect of the antibody-drug conjugate and the PARP1 selective inhibitor used in the present disclosure.

In addition, the antitumor effect of the pharmaceutical product and therapeutic method of the present disclosure can be confirmed, in a clinical study, with the Response Evaluation Criteria in Solid Tumors (RECIST) evaluation method, WHO's evaluation method, Macdonald's evaluation method, measurement of body weight, and other methods; and can be determined by indicators such as Complete response (CR), Partial response (PR), Progressive disease (PD), Objective response rate (ORR), Duration of response (DoR), Progression-free survival (PFS), and Overall survival (OS).

The foregoing methods can provide confirmation of superiority in terms of the antitumor effect of the pharmaceutical product and therapeutic method of the present disclosure compared to existing pharmaceutical products and treatment methods for cancer therapy.

The pharmaceutical product and therapeutic method of the present disclosure can retard growth of cancer cells, suppress their proliferation, and further can kill cancer cells. These effects can allow cancer patients to be free from symptoms caused by cancer or can achieve an improvement in the QOL of cancer patients and attain a therapeutic effect by sustaining the lives of the cancer patients. Even if the pharmaceutical product and therapeutic method do not accomplish the killing of cancer cells, they can achieve higher QOL of cancer patients while achieving longer-term survival, by inhibiting or controlling the growth of cancer cells.

The pharmaceutical product of the present invention can be expected to exert a therapeutic effect by application as systemic therapy to patients, and additionally, by local application to cancer tissues.

The pharmaceutical product and therapeutic method of the present disclosure, in another aspect, provides for use as an adjunct in cancer therapy with ionizing radiation or other chemotherapeutic agents. For example, in the treatment of cancer, the treatment may comprise administering to a subject in need of treatment a therapeutically-effective amount of the pharmaceutical product, simultaneously or sequentially with ionizing radiation or other chemotherapeutic agents.

The pharmaceutical product and therapeutic method of the present disclosure can be used as adjuvant chemotherapy combined with surgery operation. The pharmaceutical product of the present disclosure may be administered for the purpose of reducing tumor size before surgical operation (referred to as preoperative adjuvant chemotherapy or neoadjuvant therapy), or may be administered for the purpose of preventing recurrence of tumor after surgical operation (referred to as postoperative adjuvant chemotherapy or adjuvant therapy).

In further aspects, the pharmaceutical product of the present disclosure may be used for the treatment of cancer which is deficient in Homologous Recombination (HR) dependent DNA DSB repair activity. The HR dependent DNA DSB repair pathway repairs double-strand breaks (DSBs) in DNA via homologous mechanisms to reform a continuous DNA helix (K. K. Khanna and S. P. Jackson, Nat. Genet. 27 (3): 247-254 (2001)). The components of the HR dependent DNA DSB repair pathway include, but are not limited to, ATM (NM_000051), RAD51 (NM 002875), RAD51L1 (NM 002877), RAD51C (NM 002876), RAD51L3 (NM 002878), DMC1 (NM 007068), XRCC2 (NM 005431), XRCC3 (NM_005432), RAD52 (NM_002879), RAD54L (NM_003579), RAD54B (NM 012415), BRCA1 (NM 007295), BRCA2 (NM 000059), RAD50 (NM_005732), MRE11A (NM_005590) and NBS1 (NM_002485). Other proteins involved in the HR dependent DNA DSB repair pathway include regulatory factors such as EMSY (Hughes-Davies, et al., Cell, 115, pp 523-535). HR components are also described in Wood, et al., Science, 291, 1284-1289 (2001). A cancer which is deficient in HR dependent DNA DSB repair may comprise or consist of one or more cancer cells which have a reduced or abrogated ability to repair DNA DSBs through that pathway, relative to normal cells i.e. the activity of the HR dependent DNA DSB repair pathway may be reduced or abolished in the one or more cancer cells. The activity of one or more components of the HR dependent DNA DSB repair pathway may be abolished in the one or more cancer cells of an individual having a cancer which is deficient in HR dependent DNA DSB repair. Components of the HR dependent DNA DSB repair pathway are well characterised in the art (see for example, Wood, et al., Science, 291, 1284-1289 (2001)) and include the components listed above.

In some embodiments, the cancer cells may have a BRCA1 and/or a BRCA2 deficient phenotype i.e. BRCA1 and/or BRCA2 activity is reduced or abolished in the cancer cells. Cancer cells with this phenotype may be deficient in BRCA1 and/or BRCA2, i.e. expression and/or activity of BRCA1 and/or BRCA2 may be reduced or abolished in the cancer cells, for example by means of mutation or polymorphism in the encoding nucleic acid, or by means of amplification, mutation or polymorphism in a gene encoding a regulatory factor, for example the EMSY gene which encodes a BRCA2 regulatory factor (Hughes-Davies, et al., Cell, 115, 523-535). BRCA1 and BRCA2 are known tumor suppressors whose wild-type alleles are frequently lost in tumors of heterozygous carriers (Jasin M., Oncogene, 21 (58), 8981-93 (2002); Tutt, et al., Trends Mol Med., 8 (12), 571-6, (2002)). The association of BRCA1 and/or BRCA2 mutations with breast cancer is well-characterised in the art (Radice, P. J., Exp Clin Cancer Res., 21 (3 Suppl), 9-12 (2002)). Amplification of the EMSY gene, which encodes a BRCA2 binding factor, is also known to be associated with breast and ovarian cancer. Carriers of mutations in BRCA1 and/or BRCA2 are also at elevated risk of certain cancers, including breast, ovary, pancreas, prostate, hematological, gastrointestinal and lung cancer. In some embodiments, the individual is heterozygous for one or more variations, such as mutations and polymorphisms, in BRCA1 and/or BRCA2 or a regulator thereof. The detection of variation in BRCA1 and BRCA2 is well-known in the art and is described, for example in EP 699 754, EP 705 903, Neuhausen, S. L. and Ostrander, E. A., Genet. Test, 1, 75-83 (1992); Chappnis, P. O. and Foulkes, W. O., Cancer Treat Res, 107, 29-59 (2002); Janatova M., et al., Neoplasma, 50(4), 246-505 (2003); Jancarkova, N., Ceska Gynekol., 68{1), 11-6 (2003)). Determination of amplification of the BRCA2 binding factor EMSY is described in Hughes-Davies, et al., Cell, 115, 523-535).

Mutations and polymorphisms associated with cancer may be detected at the nucleic acid level by detecting the presence of a variant nucleic acid sequence or at the protein level by detecting the presence of a variant (i.e. a mutant or allelic variant) polypeptide.

The pharmaceutical product of the present disclosure may be administered as a pharmaceutical composition containing at least one pharmaceutically suitable ingredient. The pharmaceutically suitable ingredient can be suitably selected and applied from formulation additives or the like that are generally used in the art, in accordance with the dosage, administration concentration or the like of the antibody-drug conjugate used in the present disclosure and the PARP1 selective inhibitor. For example, the antibody-drug conjugate used in the present disclosure can be administered as a pharmaceutical composition containing a buffer such as a histidine buffer, an excipient such as sucrose or trehalose, and a surfactant such as Polysorbate 80 or 20. The pharmaceutical product containing the antibody-drug conjugate used in the present disclosure can be preferably used as an injection, can be more preferably used as an aqueous injection or a lyophilized injection, and can be even more preferably used as a lyophilized injection. In the case that the pharmaceutical product containing the anti-TROP2 antibody-drug conjugate used in the present disclosure is an aqueous injection, it can be preferably diluted with a suitable diluent and then given as an intravenous infusion. For the diluent, a dextrose solution, physiological saline, and the like, can be exemplified, and a dextrose solution can be preferably exemplified, and a 5% dextrose solution can be more preferably exemplified. In the case that the pharmaceutical product of the present disclosure is a lyophilized injection, it can be preferably dissolved in water for injection, subsequently a required amount can be diluted with a suitable diluent and then given as an intravenous infusion. For the diluent, a dextrose solution, physiological saline, and the like, can be exemplified, and a dextrose solution can be preferably exemplified, and a 5% dextrose solution can be more preferably exemplified.

Examples of the administration route which may be used to administer the pharmaceutical product of the present disclosure include intravenous, intradermal, subcutaneous, intramuscular, and intraperitoneal routes, and preferably include an intravenous route.

The anti-TROP2 antibody-drug conjugate used in the present disclosure can be administered to a human once at intervals of 1 to 180 days, and can be preferably administered once a week, once every 2 weeks, once every 3 weeks, or once every 4 weeks, and can be even more preferably administered once every 3 weeks. Also, the antibody-drug conjugate used in the present invention can be administered at a dose of about 0.001 to 100 mg/kg, and can be preferably administered at a dose of 0.8 to 12.4 mg/kg. For example, the anti-TROP2 antibody-drug conjugate can be administered once every 3 weeks at a dose of 0.27 mg/kg, 0.5 mg/kg, 1.0 mg/kg, 2.0 mg/kg, 4.0 mg/kg, 6.0 mg/kg, or 8.0 mg/kg, and can be preferably administered once every 3 weeks at a dose of 6.0 mg/kg.

The PARP1 selective inhibitor may be administered in a suitable dose by any suitable route of administration. The size of the dose required for the therapeutic treatment of a particular disease state will necessarily be varied depending on the subject treated, the route of administration and the severity of the illness being treated. For further information on routes of administration and dosage regimes, reference may be made to Chapter 25.3 in Volume 5 of Comprehensive Medicinal Chemistry (Corwin Hansch; Chairman of Editorial Board), Pergamon Press 1990.

Compounds of formula (I), or pharmaceutically acceptable salts thereof, will normally be administered via the oral route in the form of pharmaceutical preparations comprising the active ingredient or a pharmaceutically acceptable salt or solvate thereof, or a solvate of such a salt, in a pharmaceutically acceptable dosage form. Depending upon the disorder and patient to be treated, the compositions may be administered at varying doses.

The pharmaceutical formulations of the compound of formula (I) described above may be prepared for oral administration, particularly in the form of tablets or capsules, and especially involving technologies aimed at furnishing colon-targeted drug release (Patel, M. M. Expert Opin. Drug Deliv. 2011, 8 (10), 1247-1258).

The pharmaceutical formulations of the compound of formula (I) described above may conveniently be administered in unit dosage form and may be prepared by any of the methods well-known in the pharmaceutical art, for example as described in Remington's Pharmaceutical Sciences, 17th ed., Mack Publishing Company, Easton, PA., (1985).

Pharmaceutical formulations of a compound of formula (I) suitable for oral administration may comprise one or more physiologically compatible carriers and/or excipients and may be in solid or liquid form. Tablets and capsules may be prepared with binding agents, fillers, lubricants and/or surfactants, such as sodium lauryl sulfate. Liquid compositions may contain conventional additives such as suspending agents, emulsifying agents and/or preservatives. Liquid compositions may be encapsulated in, for example, gelatin to provide a unit dosage form. Solid oral dosage forms include tablets, two-piece hard shell capsules and soft elastic gelatin (SEG) capsules. Such two-piece hard shell capsules may be made for example by filling a compound of formula (I) into a gelatin or hydroxypropyl methylcellulose (HPMC) shell.

A dry shell formulation of a compound of formula (I) typically comprises of about 40% to 60% w/w concentration of gelatin, about a 20% to 30% concentration of plasticizer (such as glycerin, sorbitol or propylene glycol) and about a 30% to 40% concentration of water. Other materials such as preservatives, dyes, opacifiers and flavours also may be present. The liquid fill material comprises a solid drug that has been dissolved, solubilized or dispersed (with suspending agents such as beeswax, hydrogenated castor oil or polyethylene glycol 4000) or a liquid drug in vehicles or combinations of vehicles such as mineral oil, vegetable oils, triglycerides, glycols, polyols and surface-active agents.

Suitable daily doses of the compounds of formula (I), or a pharmaceutically acceptable salt thereof, in therapeutic treatment of humans are about 0.0001-100 mg/kg body weight. Oral formulations are preferred, particularly tablets or capsules which may be formulated by methods known to those skilled in the art to provide doses of the active compound in the range of 0.1 mg to 1000 mg.

### Examples

The present disclosure is specifically described in view of the examples shown below. However, the present disclosure is not limited to these. Further, it is by no means to be interpreted in a limited way.

### Synthesis of PARP1 Selective Inhibitors

Syntheses of exemplary PARP1 selective inhibitors are described in Examples 1 to 32 of WO 2021/013735, including the preparation of intermediate compounds, and the general experimental conditions used. In Example 4 of WO 2021/013735, 5-[4-[(7-ethyl-6-oxo-5H-1,5-naphthyridin-3-yl)methyl]piperazin-1-yl]-N-methyl-pyridine-2-carboxamide was obtained as a partially crystalline solid by evaporating a methanol/dichloromethane solution under reduced pressure. The crystalline material so-obtained was characterised as crystalline Form A, exhibiting peaks under XRPD analysis as follows:

XRPD Peaks for Form A

| Angle | Intensity |
|---|---|
| (2θ ± 0.2°) | (%) |
| 8.3 | 100.0 |
| 12.4 | 30.9 |
| 19.4 | 26.5 |
| 20.4 | 25.8 |
| 26.3 | 19.2 |
| 21.2 | 17.4 |
| 20.8 | 14.8 |
| 22.8 | 14.1 |
| 16.8 | 14.0 |
| 10.2 | 13.2 |
| 18.4 | 10.8 |
| 11.4 | 9.9 |
| 28.1 | 8.4 |
| 18.0 | 8.4 |
| 25.2 | 8.2 |
| 24.9 | 6.7 |
| 16.5 | 6.4 |
| 17.3 | 5.3 |
| 22.1 | 4.0 |
| 29.3 | 3.3 |
| 24.3 | 2.7 |
| 30.3 | 2.5 |
| 38.2 | 2.0 |
| 33.9 | 1.4 |
| 14.2 | 1.4 |
| 13.7 | 1.4 |
| 33.0 | 1.3 |
| 36.5 | 1.2 |
| 39.2 | 1.2 |

Form A is characterized in providing at least one of the following 2θ values measured using CuKα radiation: 8.3, 12.4, and 19.4°. DSC analysis indicates that Form A has a melting point with an onset at 254° C. and a peak at 255° C.

### Biological Assays for PARP1 Selective Inhibitors

Test procedures as described in WO 2021/013735 (PARP Fluorescence Anisotropy binding assays; hERG Electrophysiological Assay; PARP Proliferation Assay-4 day compound dosing) may be employed to determine the inhibitory properties of PARP1 selective inhibitor compounds described herein.

Analysis results for PARP1 selective inhibitors as described in Examples 1 to 32 of WO 2021/013735 are shown as follows:

| | | | | | | BRCA2 −/− | WT | |
|---|---|---|---|---|---|---|---|---|
| | | | | | | DLD-1 | DLD-1 | |
| | | | | | | prolif | prolif | |
| | PARP1 | PARP2 | PARP3 | PARP5a | PARP6 | 4 d | 4 d | hERG |
| Example | IC50 | IC50 | IC50 | IC50 | IC50 | IC50 | IC50 | IC50 |
| No. | (μM) | (μM) | (μM) | (μM) | (μM) | (μM) | (μM) | (μM) |
| 1 | 0.003 | 1.7 | 4 | >100 | 34 | 0.010 | >30 | >40 |
| 2 | 0.004 | 0.88 | 9.9 | 20 | 14 | 0.008 | >30 | >40 |
| 3 | 0.005 | 1.3 | 12 | >100 | 14 | 0.004 | >30 | 22 |
| 4 | 0.004 | >1.5 | 4.7 | >100 | 19 | >0.017 | >30 | >40 |
| 5 | 0.002 | 0.65 | 7.1 | >100 | 23 | 0.006 | >30 | >40 |
| 6 | 0.003 | 0.84 | 9.3 | >100 | 8.2 | 0.006 | >30 | >40 |
| 7 | 0.002 | 1.3 | 2.6 | 94 | 22 | 4.14 | | |
| 8 | 0.003 | 11 | 55 | 93 | 18 | 0.011 | >19 | >40 |
| 9 | 0.009 | 22 | >100 | >100 | 47 | 0.010 | 17 | >40 |
| 10 | 0.005 | 17 | 48 | 56 | 26 | 0.006 | >30 | >40 |
| 11 | 0.005 | 4 | 13 | >100 | 22 | 0.184 | >30 | >40 |
| 12 | 0.004 | 1.6 | 19 | 89 | 11 | 0.008 | >30 | >40 |
| 13 | 0.007 | 8.5 | 30 | >100 | 30 | 0.005 | >26 | >40 |
| 14 | 0.004 | 2.9 | 30 | 50 | 11 | 0.006 | >30 | >40 |
| 15 | 0.011 | 3.6 | 35 | >100 | 80 | 0.090 | >30 | >40 |
| 16 | 0.007 | 3.3 | 74 | 61 | 31 | 0.018 | >22 | >40 |
| 17 | 0.007 | 1.7 | 96 | >100 | 59 | 0.020 | >30 | >40 |
| 18 | 0.031 | 17 | >100 | >100 | >29 | 4.90 | >30 | 5.2 |
| 19 | 0.015 | >100 | >100 | >100 | >29 | 0.015 | >30 | 21 |
| 20 | 0.014 | 28 | >100 | >100 | >100 | 0.016 | >24 | 38 |
| 21 | 0.004 | 9.5 | >100 | >100 | 33 | 0.016 | >30 | >40 |
| 22 | 0.006 | 1 | 2.6 | 26 | 16 | 0.012 | >30 | >40 |
| 23 | 0.004 | 4.4 | 60 | 60 | >100 | | 4.2 | 36 |
| 24 | 0.003 | 5.1 | >100 | 93 | >100 | 0.010 | 14 | 37 |
| 25 | 0.002 | 6 | 43 | >100 | >100 | | >25 | >40 |
| 26 | 0.005 | 6.7 | >100 | >100 | >100 | 0.005 | 23 | >40 |
| 27 | 0.007 | 16 | >100 | 71 | >100 | 10.3 | >10 | 26 |
| 28 | 0.006 | 14 | >100 | >29 | >100 | 0.027 | >30 | >40 |
| 29 | 0.004 | 6.1 | 9.9 | >100 | 14 | 0.007 | >30 | >40 |
| 30 | 0.003 | 7.6 | 4.5 | >100 | 10 | 0.004 | >30 | >40 |
| 31 | 0.005 | 3.7 | 2.6 | >100 | 28 | | | >40 |
| 32 | 0.003 | 2.1 | 1.9 | >100 | 10 | | | >40 |

### Example 1: Production of Anti-TROP2 Antibody-Drug Conjugate

In accordance with a production method described in WO 2015/098099 and WO 2017/002776 and using an anti-TROP2 antibody (an antibody comprising a heavy chain consisting of an amino acid sequence represented by SEQ ID NO: 12 [=amino acid residues 20 to 470 of SEQ ID NO: 1] and a light chain consisting of an amino acid sequence represented by SEQ ID NO: 13 [=amino acid residues 21 to 234 of SEQ ID NO: 2]), an anti-TROP2 antibody-drug conjugate in which a drug-linker represented by the following formula:

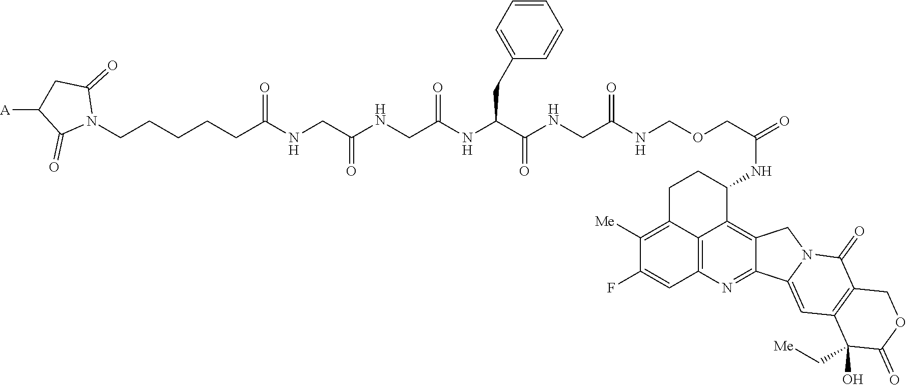

wherein A represents the connecting position to an antibody, is conjugated to the anti-TROP2 antibody via a thioether bond was produced (DS-1062: datopotamab deruxtecan). The DAR of the antibody-drug conjugate is ˜4.

### Example 2: Production of PARP1 Selective Inhibitor

In accordance with the method of Example 4 of WO 2021/013735, a PARP1 selective inhibitor of formula (I), specifically 5-[4-[(7-ethyl-6-oxo-5H-1,5-naphthyridin-3-yl)methyl]piperazin-1-yl]-N-methyl-pyridine-2-carboxamide (AZD5305), was produced:

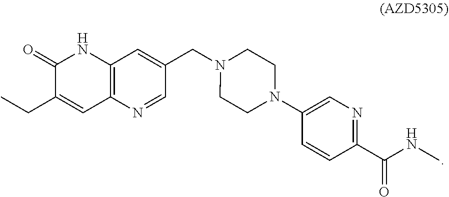 [SMILES: CCc1cc2ncc(CN3CCN(c4ccc(C(=O)NC)nc4)CC3)cc2[nH]c1=O]

### Example 3: Antitumor Test

Combination of antibody-drug conjugate DS-1062 (datopotamab deruxtecan) with PARP1 selective inhibitor AZD5305 (5-[4-[(7-ethyl-6-oxo-5H-1,5-naphthyridin-3-yl)methyl]piperazin-1-yl]-N-methyl-pyridine-2-carboxamide) or olaparib

### Method:

A high-throughput combination screen was run, in which 6 lung and 5 breast cancer cell lines (Table 1) were treated with combinations of DS-1062 and AZD5305 (PARP1 selective inhibitor) or olaparib.

TABLE 1

| Cell Line | Cancer Type |
|---|---|
| NCI-H1975 | Lung |
| NCI-H1650 | Lung |
| NC-H322 | Lung |
| HCC2935 | Lung |
| NCI-H3255 | Lung |
| CALU3 | Lung |
| HCC70 | Breast |
| HCC1187 | Breast |
| HCC1806 | Breast |
| MDA-MB-468 | Breast |
| HCC38 | Breast |

The readout of the screen was a 7-day CellTiter-Glo cell viability assay, conducted as a 6×6 dose response matrix (5-point log serial dilution for DS-1062, and half log serial dilution for AZD5305 or olaparib). Maximum concentration was 3 μM for AZD5305 and olaparib, and 10 μg/ml for DS-1062. In addition, exatecan (DNA topoisomerase I inhibitor) was also screened in parallel with AZD5305, to help deconvolute the mechanism of action of effective combinations. Combination activity was assessed based on a combination of the Combination Emax and Loewe synergy scores.

### Results:

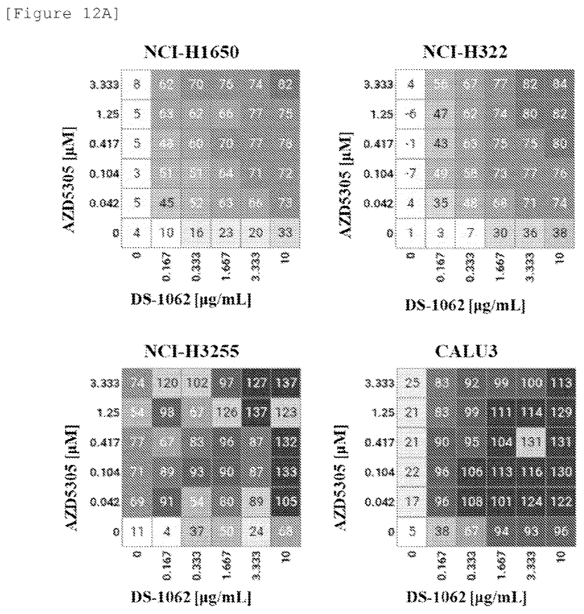

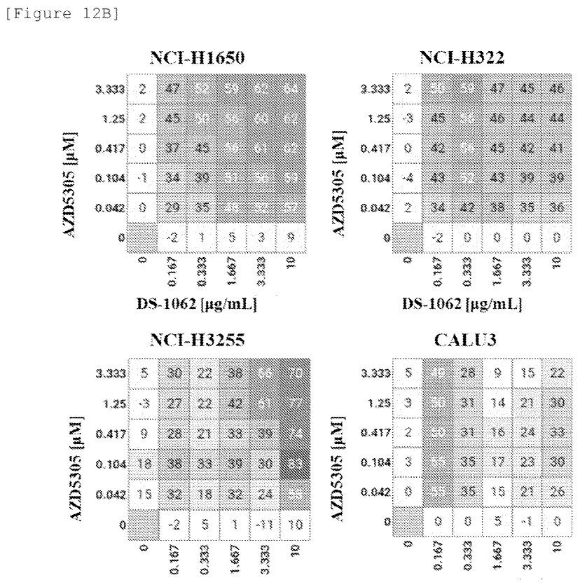

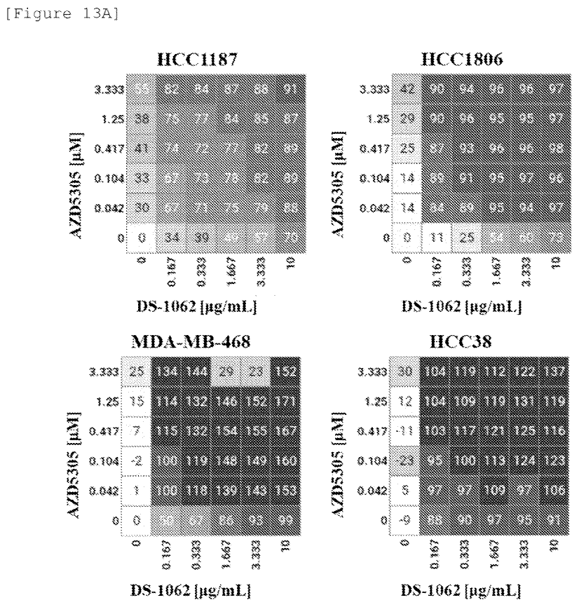

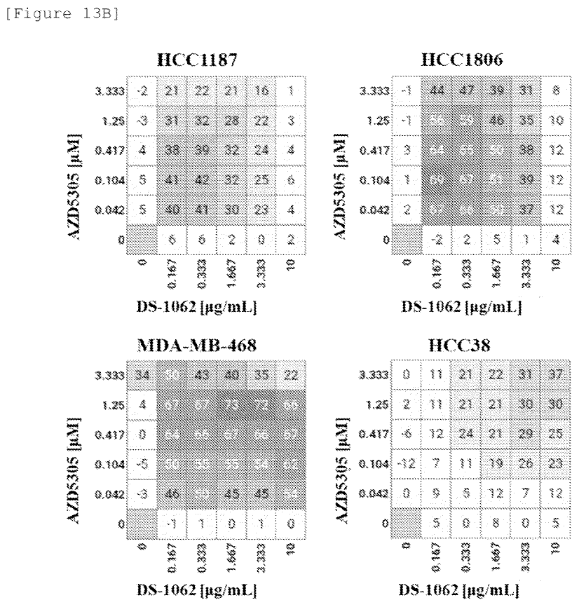

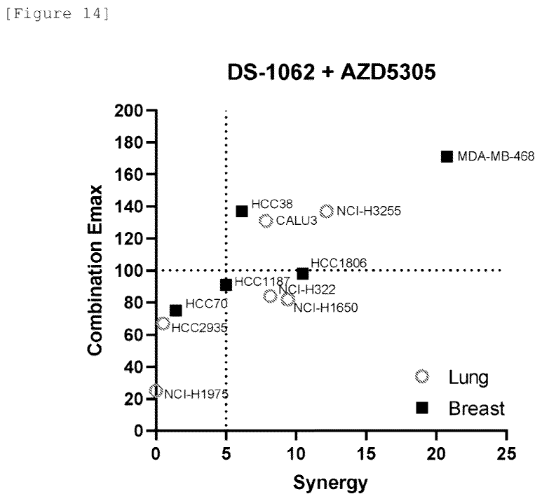

Results for the DS-1062+AZD5305 combination are shown for TROP2-expressing lung cancer cell lines (NCIH1650, NCI-H322, NCI-H3255, CALU3) in FIGS. 12A and 12B and Table 2, and for TROP2-expressing breast cancer cell lines (HCC1187, HCC1806, MDA-MB-468, HCC38) in FIGS. 13A and 13B and Table 3.

FIGS. 12A and 13A show matrices of measured cell viability signals. X axes represent drug A (DS-1062), and Y axes represent drug B (AZD5305). Values in the box represent the ratio of cells treated with drug A+B compared to DMSO control at day 7. All values are normalised to cell viability values at day 0. Values between 0 and 100 represent % growth inhibition and values above 100 represent cell death.

FIGS. 12B and 13B show Loewe excess matrices. Values in the box represent excess values calculated by the Loewe additivity model.

Tables 2 and 3 show HSA and Loewe additivity scores and Combination Emax:

TABLE 2
DS-1062 + AZD5305 Combination
Results in Lung Cancer Cell Lines

| Cell line | NCI-H1650 | NCI-H322 | NCI-H3255 | CALU3 |
|---|---|---|---|---|
| HSA synergy | 9.43 | 8.14 | 12.23 | 7.84 |
| score | | | | |
| Loewe synergy | 9.43 | 8.14 | 12.18 | 7.84 |
| score | | | | |
| Combination | 82 | 84 | 137 | 131 |
| Emax | | | | |

TABLE 3
DS-1062 + AZD5305 Combination
Results in Breast Cancer Cell Lines

| Cell line | HCC1187 | HCC1806 | MDA-MB-468 | HCC38 |
|---|---|---|---|---|
| HSA synergy | 5.69 | 11.16 | 20.74 | 6.19 |
| score | | | | |
| Loewe synergy | 5.00 | 10.46 | 20.74 | 6.13 |
| score | | | | |
| Combination | 91 | 98 | 171 | 137 |
| Emax | | | | |

### Notes:

Loewe Dose Additivity predicts the expected response if the two compounds act on the same molecular target by means of the same mechanism. It calculates additivity based on the assumption of zero interaction between the compounds and it is independent from the nature of the dose-response relationship.
HSA (Highest Single Agent) [Berenbaum 1989] quantifies the higher of the two single compound effects at their corresponding concentrations. The combined effect is compared with the effect of each single agent at the concentration used in the combination. Excess over the highest single agent effect indicates cooperativity. HSA does not require the compounds to affect the same target.
Excess Matrix: For each well in the concentration matrix, the measured or fitted values are compared to the predicted non-synergistic values for each concentration pair. The predicted values are determined by the chosen model. Differences between the predicted and observed values may indicate synergy or antagonism, and are shown in the Excess Matrix. Excess Matrix values are summarized by the combination scores Excess Volume and Synergy Score.
Combination Emax: The maximum anti-proliferative effect observed in the combination matrix tested. All values are normalised to cell viability values at day 0. Values between 0 and 100 represent % growth inhibition and values above 100 represent cell death.

FIG. 14 shows combination Emax and Loewe synergy scores in various cell lines treated with DS-1062 combined with AZD5305.

As seen from FIGS. 12A and 12B, and Table 2, AZD5305 interacted synergistically with DS-1062 and also increased cell death in TROP2-expressing lung cancer cell lines. As seen from FIGS. 13A and 13B, and Table 3, AZD5305 interacted synergistically with DS-1062 and also increased cell death in TROP2-expressing breast cancer cell lines at Emax (3 μM AZD5305 and 10 μg/ml DS-1062).

As seen from FIG. 14, in four cell lines, treatment with DS-1062 combined with AZD5305 resulted in high combination Emax (>100) and high Loewe synergy scores (>5).

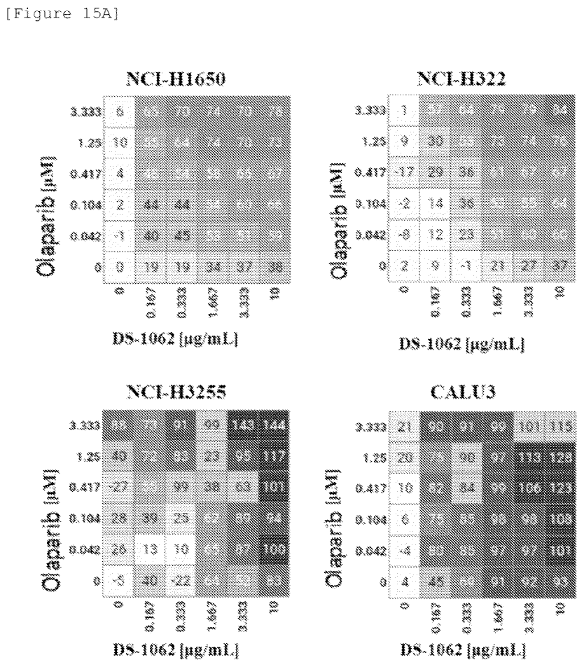

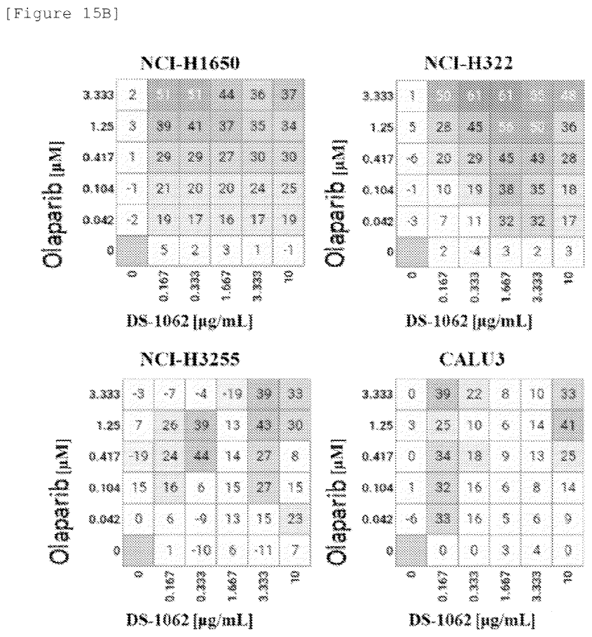

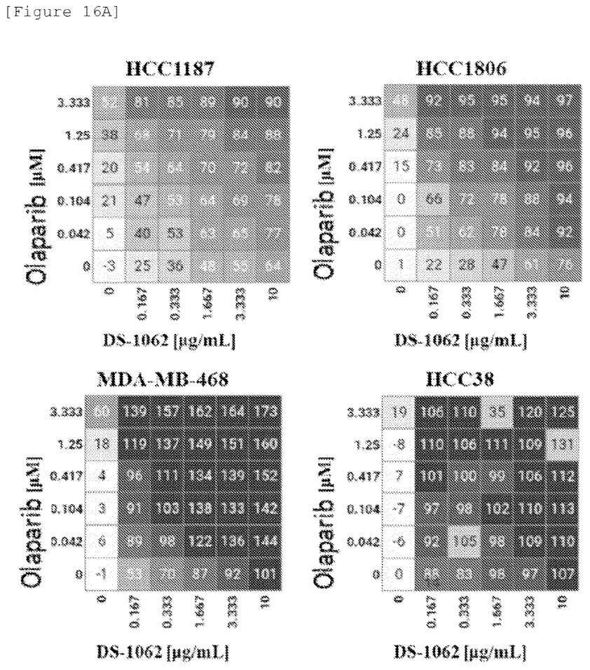

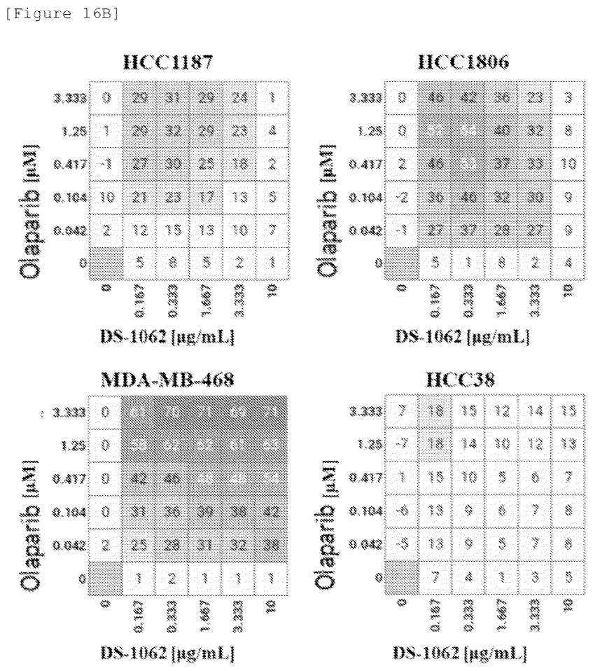

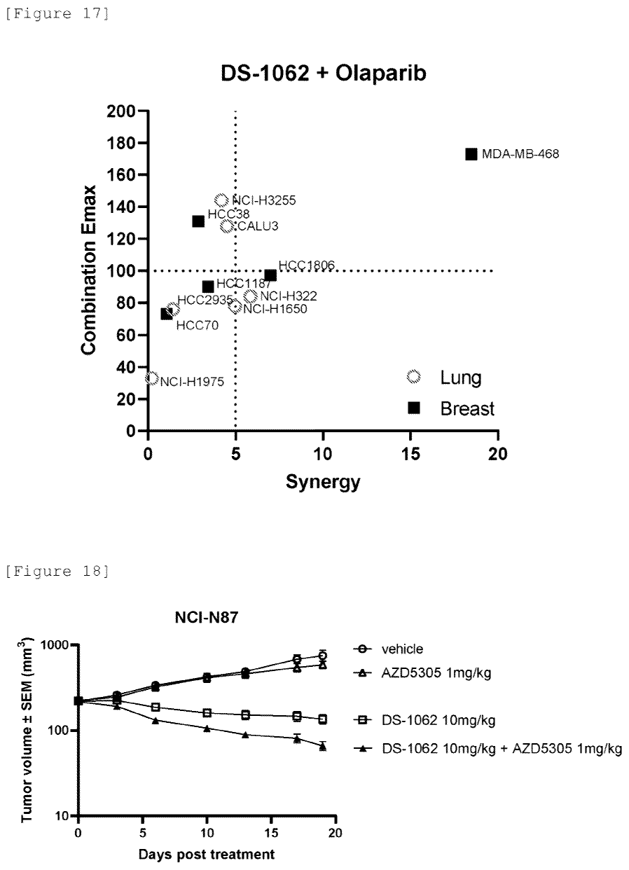

Results for the DS-1062+olaparib combination are shown for TROP2-expressing lung cancer cell lines (NCIH1650, NCI-H322, NCI-H3255, CALU3) in FIGS. 15A and 15B and Table 4, and for TROP2-expressing breast cancer cell lines (HCC1187, HCC1806, MDA-MB-468, HCC38) in FIGS. 16A and 16B and Table 5.

FIGS. 15A and 16A show matrices of measured cell viability signals. X axes represent drug A (DS-1062), and Y axes represent drug B (olaparib). Values in the box represent the ratio of cells treated with drug A+B compared to DMSO control at day 7. All values are normalised to cell viability values at day 0. Values between 0 and 100 represent % growth inhibition and values above 100 represent cell death.

FIGS. 15B and 16B show Loewe excess matrices. Values in the box represent excess values calculated by the Loewe additivity model.

Tables 4 and 5 show HSA and Loewe additivity scores and Combination Emax:

TABLE 4
DS-1062 + Olaparib Combination
Results in Lung Cancer Cell Lines

| Cell line | NCI-H1650 | NCI-H322 | NCI-H3255 | CALU3 |
|---|---|---|---|---|
| HSA synergy | 4.67 | 5.85 | 5.55 | 4.50 |
| score | | | | |
| Loewe synergy | 4.97 | 5.85 | 4.19 | 4.50 |
| score | | | | |
| Combination | 78 | 84 | 144 | 128 |
| Emax | | | | |

TABLE 5
DS-1062 + Olaparib Combination
Results in Breast Cancer Cell Lines

| Cell line | HCC1187 | HCC1806 | MDA-MB-468 | HCC38 |
|---|---|---|---|---|
| HSA synergy | 4.10 | 7.90 | 19.55 | 2.88 |
| score | | | | |
| Loewe synergy | 3.44 | 7.00 | 18.47 | 2.88 |
| score | | | | |
| Combination | 90 | 97 | 173 | 131 |
| Emax | | | | |

FIG. 17 shows combination Emax and Loewe synergy scores in various cell lines treated with DS-1062 combined with olaparib.

As seen from FIGS. 15A and 15B, and Table 4, olaparib interacted synergistically with DS-1062 and also increased cell death in TROP2-expressing lung cancer cell lines. As seen from FIGS. 16A and 16B, and Table 5, olaparib interacted synergistically with DS-1062 and also increased cell death in TROP2-expressing breast cancer cell lines at Emax (3 μM olaparib and 10 μg/ml DS-1062). As seen from FIG. 17, in one cell line, treatment with DS-1062 combined with olaparib resulted in high combination Emax (>100) and high Loewe synergy scores (>5).

The results in Example 3 demonstrate that selective PARP1 inhibition using AZD5305 or olaparib enhances the antitumor efficacy of DS-1062 in TROP2-expressing lung and breast cancer cell lines in vitro. In Example 3, AZD5305 in combination with DS-1062 showed combination benefit in four TROP2-expressing lung cancer cell lines (FIGS. 12A, 12B, 14 and Table 2) and four TROP2-expressing breast cancer cell lines (FIGS. 13A, 13B and 14, and Table 3). The DS-1062+AZD5305 combination shows greater synergy than the DS-1062+Olaparib combination in particular cell lines (e.g. NCI-H1650, NCI-H3255, HCC1806, HCC38).

### Example 4: Antitumor Test—In Vivo—NCI-N87 Xenograft Model

Combination of antibody-drug conjugate DS-1062 (datopotamab deruxtecan) with PARP1 selective inhibitor AZD5305 (5-[4-[(7-ethyl-6-oxo-5H-1,5-naphthyridin-3-yl)methyl]piperazin-1-yl]-N-methyl-pyridine-2-carboxamide)

### Method:

Female Nude mice (Charles River) aged 5-8 weeks were used, following 7 days acclimatisation before entry into the study. 5×10⁶ NCI-N87 tumor cells (gastric cancer cell line) (1:1 in Matrigel) were implanted subcutaneously onto the flank of the female Nude mice. When tumors reached approximately 250 mm³, similar-sized tumors were randomly assigned to treatment groups as shown in Table 6:

TABLE 6

| | | | Route of | Dosing Schedule |
|---|---|---|---|---|
| Treatment | Dose | | administration | (28 days) |
| Vehicle | — | | IV + PO | Single dose + QD |
| DS-1062 | 10 | mg/kg | IV | Single dose |
| AZD5305 | 1 | mg/kg | PO | QD |

| | | | |
|---|---|---|---|
| DS-1062 + | 10 mg/kg + | IV + PO | Single dose + QD |
| AZD5305 | 1 mg/kg | | |

PO: oral (per os) dosing
QD: once per day (quaque die) dosing

The dose of compound for each animal was calculated based on the individual body weight on the day of dosing. DS-1062 and AZD5305 were dosed on the same day, with DS-1062 being administered approximately 5 hour post the PO dose of AZD5305. DS-1062 was administered as a single dose at 10 mg/kg on day 1, and AZD5305 was administered at 1 mg/kg QD for 21 days. Duration of dosing was for 21 days.

Formulation of DS-1062 at 10 mg/kg

The dosing solutions for DS-1062 were prepared on the day of dosing by diluting the DS-1062 stock (20.1 mg/ml) in 25 mM histidine buffer, 9% sucrose (pH5.5) to 0.6 mg/ml, and 2 mg/ml for the 3 mg/kg and 10 mg/kg dosing solutions, respectively. Each dosing solution was mixed well using a pipette before administration via IV injection at a dosing volume of 5 ml/kg.

Formulation of AZD5305 at 1 mg/kg

To formulate for a 1 mg/kg dosing solution, a concentration of 0.1 mg/ml AZD5305 was prepared which resulted in a dosing volume of 10 ml/kg for PO dosing. A total of 49 ml of vehicle was required. A volume of 15 μl of 1M HCl was added to the compound and mixed well by vortexing. A volume of 1 ml of sterile water was added to the Eppendorf tube and mixed well with the compound using a pellet pestle. The compound was sonicated for approximately 5 minutes then the contents transferred to a glass bottle. A volume of 1 ml sterile water was used to rinse the Eppendorf tube of any remaining compound and was then transferred to the glass bottle. The remaining volume of sterile water (37.2 ml; a total of 80% of the total vehicle volume) was added to the glass bottle and mixed well using a magnetic stirrer. The pH of the dosing solution was adjusted to pH 3.74 then the remaining vehicle (9.772 ml of sterile water) was added to the glass bottle and mixed well using a magnetic stirrer. The dosing solution was protected from light and a small aliquot was taken daily for dosing. All remaining dosing solution was kept for up to 7 days in the fridge. The final dosing matrix for 1 mg/kg AZD5305 was a clear solution.

### Measurements

Tumor growth inhibition (TGI) was calculated as follows:

$$TGI \% = \{ 1 - ( MTV treated / MTV control ) \} * 100$$

where MTV=mean tumor volume.

Statistical significance was evaluated using one-tailed t-test of (log (relative tumor volume)=log (final vol/start vol)) at the day of final measure, comparing to vehicle control.

### Results

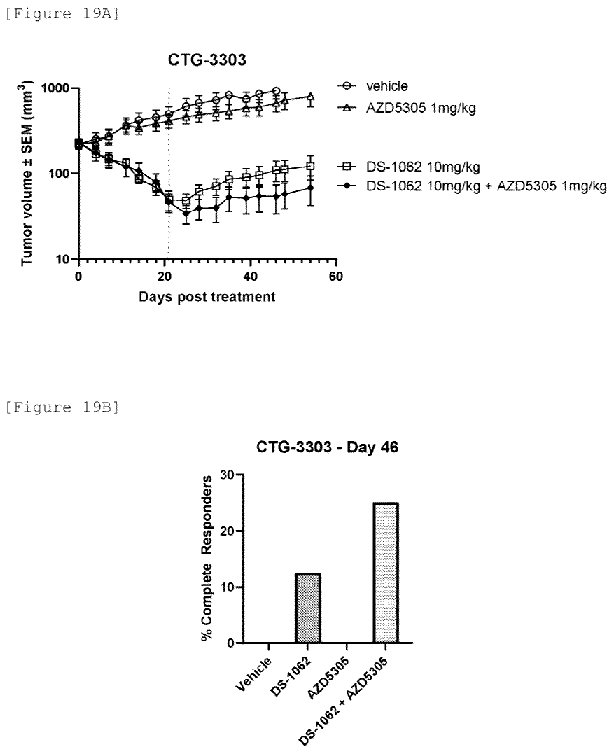

Tumor volumes for treatments with DS-1062 or AZD5305 alone or with DS-1062 in combination with AZD5305 are shown in FIG. 18. Data represents change in tumor volume over time for treatment groups. The dotted line in FIG. 18 represents end of dosing periods. For full dose and schedule information, refer to Table 6 above. Values shown are mean±SEM; n=8 for all treatment groups.

TGI responses (Day 19 TGI %) following treatment with DS-1062 or AZD5305 alone or with DS-1062 in combination with AZD5305, in NCI-N87 xenograft, are shown in Table 7:

TABLE 7

| | TGI % | p-value vs | |
|---|---|---|---|
| Treatment group | day 19 | vehicle | Significance |
| DS-1062 10 mg/kg | 82% | 0.0023 | ** |
| AZD5305 1 mg/kg | 22% | 0.7104 | †ns |
| DS-1062 10 mg/kg + | 91% | 0.0006 | *** |
| AZD5305 1 mg/kg | | | |

†not significant

Monotherapy with DS-1062 at 10 mg/kg showed TGI value of 82% at day 19 post treatment. AZD5305 monotherapy achieved a TGI of 22% at day 19 post treatment. Combination treatment of AZD5305 with DS-1062 at 10 mg/kg resulted in a TGI of 91% at 19 days post treatment and showed better response than either respective monotherapies.

Treatment groups were generally well tolerated and average bodyweights of all treatment groups remained stable during the study.

### Example 5: Antitumor Test—In Vivo—TNBC Patient Derived Xenograft Model

Combination of antibody-drug conjugate DS-1062 (datopotamab deruxtecan) with PARP1 selective inhibitor AZD5305 (5-[4-[(7-ethyl-6-oxo-5H-1,5-naphthyridin-3-yl)methyl]piperazin-1-yl]-N-methyl-pyridine-2-carboxamide)

### Method:

Female Nude mice (Charles River) aged 5-8 weeks were used, following 7 days acclimatisation before entry into the study. The human patient-derived xenograft (PDX) model, CTG-3303, was established from fragments of freshly resected tumor of a triple negative breast cancer (TNBC) patient whom relapsed on treatment with PARP inhibitor talazoparib. This PDX was obtained in accordance with appropriate consent procedures. This TNBC PDX model was subcutaneously passaged in vivo as fragments from animal to animal in nude mice. When tumors reached approximately 250 mm³, similar-sized tumors were randomly assigned to treatment groups as shown in Table 8:

TABLE 8

| | | | Route of | Dosing Schedule |
|---|---|---|---|---|
| Treatment | Dose | | administration | (28 days) |
| Vehicle | — | | IV + PO | Single dose + QD |
| DS-1062 | 10 | mg/kg | IV | Single dose |
| AZD5305 | 1 | mg/kg | PO | QD |

| | | | |
|---|---|---|---|
| DS-1062 + | 10 mg/kg + | IV + PO | Single dose + QD |
| AZD5305 | 1 mg/kg | | |

PO: oral (per os) dosing
QD: once per day (quaque die) dosing

The dose of compound for each animal was calculated based on the individual body weight on the day of dosing. DS-1062 and AZD5305 were dosed on the same day, with DS-1062 being administered approximately 5 hour post the PO dose of AZD5305. DS-1062 was administered as a single dose at 10 mg/kg on day 1, and AZD5305 was administered at 1 mg/kg QD for 21 days. Duration of dosing was for 21 days.

Formulation of DS-1062 at 10 mg/kg

The dosing solutions for DS-1062 were prepared on the day of dosing by diluting the DS-1062 stock (20.1 mg/ml) in 25 mM histidine buffer, 9% sucrose (pH5.5) to 0.6 mg/ml, and 2 mg/ml for the 3 mg/kg and 10 mg/kg dosing solutions, respectively. Each dosing solution was mixed well using a pipette before administration via IV injection at a dosing volume of 5 ml/kg.

Formulation of AZD5305 at 1 mg/kg

To formulate for a 1 mg/kg dosing solution, a concentration of 0.1 mg/ml AZD5305 was prepared which resulted in a dosing volume of 10 ml/kg for PO dosing. A total of 49 ml of vehicle was required. A volume of 15 μl of 1M HCl was added to the compound and mixed well by vortexing. A volume of 1 ml of sterile water was added to the Eppendorf tube and mixed well with the compound using a pellet pestle. The compound was sonicated for approximately 5 minutes then the contents transferred to a glass bottle. A volume of 1 ml sterile water was used to rinse the Eppendorf tube of any remaining compound and was then transferred to the glass bottle. The remaining volume of sterile water (37.2 ml; a total of 80% of the total vehicle volume) was added to the glass bottle and mixed well using a magnetic stirrer. The pH of the dosing solution was adjusted to pH 3.74 then the remaining vehicle (9.772 ml of sterile water) was added to the glass bottle and mixed well using a magnetic stirrer. The dosing solution was protected from light and a small aliquot was taken daily for dosing. All remaining dosing solution was kept for up to 7 days in the fridge. The final dosing matrix for 1 mg/kg AZD5305 was a clear solution.

### Measurements

Tumor growth inhibition (TGI) was calculated as follows:

$$TGI \% = \{ 1 - ( MTV treated / MTV control ) \} * 100$$

where MTV=mean tumor volume.

Statistical significance was evaluated using one-tailed t-test of (log (relative tumor volume)=log (final vol/start vol)) at the day of final measure, comparing to vehicle control.

### Results

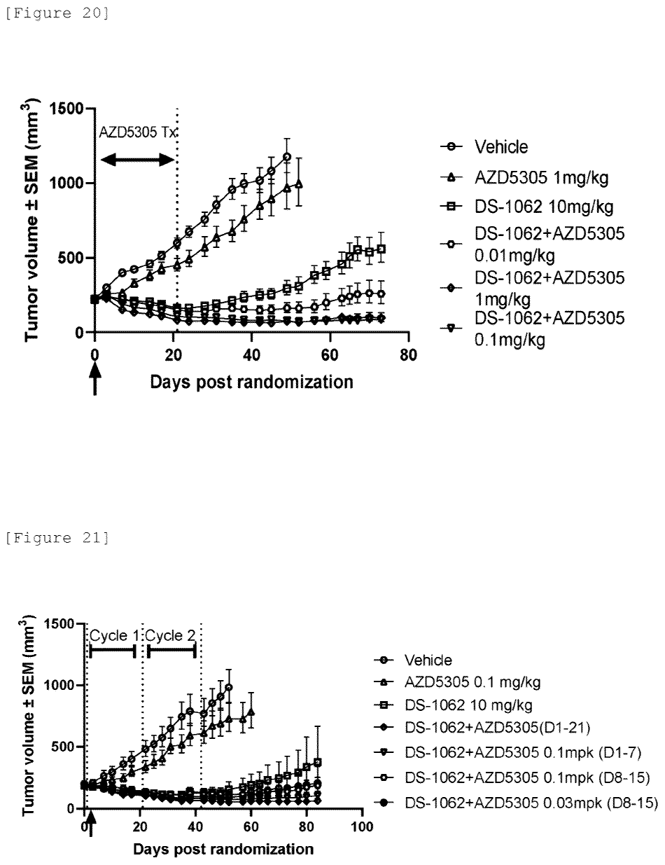

Tumor volumes for treatments with DS-1062 or AZD5305 alone or with DS-1062 in combination with AZD5305 are shown in FIG. 19A. Data represents change in tumor volume over time for treatment groups. The dotted line in FIG. 19A represents end of dosing periods. For full dose and schedule information, refer to Table 8 above. Values shown are mean±SEM; n=8 for all treatment groups.

TGI responses (Day 46 TGI %) following treatment with DS-1062 or AZD5305 alone or with DS-1062 in combination with AZD5305, in NCI-N87 xenograft, are shown in Table 9:

TABLE 9

| | TGI % | p-value vs | |
|---|---|---|---|
| Treatment group | day 46 | vehicle | Significance |
| DS-1062 10 mg/kg | 88% | 0.0001 | **** |
| AZD5305 1 mg/kg | 28% | 0.338 | †ns |
| DS-1062 10 mg/kg + | 94% | 0.0001 | **** |
| AZD5305 1 mg/kg | | | |

†not significant

Monotherapy with DS-1062 at 10 mg/kg showed TGI value of 88% at day 46 post treatment. AZD5305 monotherapy achieved a TGI of 28% at day 46 post treatment. Combination treatment of AZD5305 with DS-1062 at 10 mg/kg resulted in a TGI of 94% at 94 days post treatment and showed better response than either respective monotherapy.

Treatment groups were generally well tolerated and average bodyweights of all treatment groups remained stable during the study.

In addition, the percentage of mice in each treatment group achieving a complete response (defined as tumor volume <20 mm³) at day 46 post treatment were calculated and shown in FIG. 19B. Monotherapy with DS-1062 at 10 mg/kg led to 1 out 8 mice (12.5%) achieving a complete response at day 46 post treatment. AZD5305 monotherapy led to 0 out 8 mice (0%) achieving a complete response at day 46 post treatment. Combination treatment of AZD5305 with DS-1062 at 10 mg/kg led to 2 out 8 mice (25%) achieving a complete response at day 46 post treatment and led to higher complete response rates than either respective monotherapy.

### Example 6: Antitumor Test (Dose Titration)—In Vivo—NCI-N87 Xenograft Model

Combination of antibody-drug conjugate DS-1062 (datopotamab deruxtecan) with PARP1 selective inhibitor AZD5305 (5-[4-[(7-ethyl-6-oxo-5H-1,5-naphthyridin-3-yl)methyl]piperazin-1-yl]-N-methyl-pyridine-2-carboxamide)-dose titration

### Method:

Female Nude mice (Charles River) aged 5-8 weeks were used, following 7 days acclimatisation before entry into the study. 5×10⁶ NCI-N87 tumor cells (gastric cancer cell line) (1:1 in Matrigel) were implanted subcutaneously onto the flank of the female Nude mice. When tumors reached approximately 250 mm³, similar-sized tumors were randomly assigned to treatment groups as shown in Table 10:

TABLE 10

| | | | Route of | Dosing Schedule |
|---|---|---|---|---|
| Treatment | Dose | | administration | (21 days) |
| Vehicle | — | | IV + PO | Single dose + QD |
| DS-1062 | 10 | mg/kg | IV | Single dose |
| AZD5305 | 1 | mg/kg | PO | QD |

| | | | |
|---|---|---|---|
| DS-1062 + | 10 mg/kg + | IV + PO | Single dose + QD |
| AZD5305 | 1 mg/kg | | |
| DS-1062 + | 10 mg/kg + | IV + PO | Single dose + QD |
| AZD5305 | 0.1 mg/kg | | |
| DS-1062 + | 10 mg/kg + | IV + PO | Single dose + QD |
| AZD5305 | 0.01 mg/kg | | |

PO: oral (per os) dosing
QD: once per day (quaque die) dosing

The dose of compound for each animal was calculated based on the individual body weight on the day of dosing. DS-1062 and AZD5305 were dosed on the same day, with DS-1062 being administered approximately 5 hour post the PO dose of AZD5305. DS-1062 was administered as a single dose at 10 mg/kg on day 1, and AZD5305 was administered at 1, 0.1 or 0.01 mg/kg QD for 21 days. Duration of dosing was for 21 days.

### Results

Tumor volumes for treatments with DS-1062 or AZD5305 alone or with DS-1062 in combination with AZD5305 are shown in FIG. 20. Data represents change in tumor volume over time for treatment groups. The dotted line in FIG. 20 represents end of dosing periods. For full dose and schedule information, refer to Table 10 above. Values shown are mean±SEM; n=8 for all treatment groups.

TGI responses (Day 49 TGI %) following treatment with DS-1062 or AZD5305 alone or with DS-1062 in combination with AZD5305, in NCI-N87 xenograft, are shown in Table 11:

TABLE 11

| | TGI % | p-value vs | |
|---|---|---|---|
| Treatment group | day 49 | vehicle | Significance |
| DS-1062 10 mg/kg | 75% | 0.0001 | **** |
| AZD5305 1 mg/kg | 18% | 0.2475 | †ns |
| DS-1062 10 mg/kg + | 94% | 0.0001 | **** |
| AZD5305 1 mg/kg | | | |
| DS-1062 10 mg/kg + | 93% | 0.0001 | **** |
| AZD5305 0.1 mg/kg | | | |
| DS-1062 10 mg/kg + | 86% | 0.0001 | **** |
| AZD5305 0.01 mg/kg | | | |

†not significant

Monotherapy with DS-1062 at 10 mg/kg showed TGI value of 75% at day 49 post treatment. AZD5305 monotherapy achieved a TGI of 18% at day 49 post treatment. Combination treatment of DS-1062 with AZD5305 at 1 mg/kg resulted in a TGI of 94% at 49 days post treatment, while combination with AZD5305 at 0.1 mg/kg resulted in a TGI of 93% at 49 days post treatment, indicating that lowering the dose of AZD5305 does not lower the combination efficacy.

Treatment groups were generally well tolerated and average bodyweights of all treatment groups remained stable during the study.

### Example 7: Antitumor Test (Dose Regimen Optimization)—In Vivo—NCI-N87 Xenograft Model

Combination of antibody-drug conjugate DS-1062 (datopotamab deruxtecan) with PARP1 selective inhibitor AZD5305 (5-[4-[(7-ethyl-6-oxo-5H-1,5-naphthyridin-3-yl)methyl]piperazin-1-yl]-N-methyl-pyridine-2-carboxamide)-dose regimen optimization

### Method:

Female Nude mice (Charles River) aged 5-8 weeks were used, following 7 days acclimatisation before entry into the study. 5×10⁶ NCI-N87 tumor cells (gastric cancer cell line) (1:1 in Matrigel) were implanted subcutaneously onto the flank of the female Nude mice. When tumors reached approximately 250 mm³, similar-sized tumors were randomly assigned to treatment groups as shown in Table 12:

TABLE 12

| | | | Route of | |
|---|---|---|---|---|
| Treatment | Dose | | administration | Dosing Schedule |
| Vehicle | — | | IV + PO | Single dose + QD |
| DS-1062 | 10 | mg/kg | IV | Single dose |
| AZD5305 | 1 | mg/kg | PO | QD (21 days) |

| | | | |
|---|---|---|---|
| DS-1062 + | 10 mg/kg + | IV + PO | Single dose + QD |
| AZD5305 | 0.1 mg/kg | | (21 days) |
| DS-1062 + | 10 mg/kg + | IV + PO | Single dose + QD |
| AZD5305 | 0.1 mg/kg | | (Day 1-7) |
| DS-1062 + | 10 mg/kg + | IV + PO | Single dose + QD |
| AZD5305 | 0.1 mg/kg | | (Day 8-15) |
| DS-1062 + | 10 mg/kg + | IV + PO | Single dose + QD |
| AZD5305 | 0.03 mg/kg | | (Day 8-15) |

PO: oral (per os) dosing
QD: once per day (quaque die) dosing

The dose of compound for each animal was calculated based on the individual body weight on the day of dosing. DS-1062 was administered as a single dose at 10 mg/kg on day 1, and AZD5305 was administered at 0.1 mg/kg QD from Day 1-21, from Day 1-7 or from Day 8-15 or at 0.03 mg/kg QD from Day 8-15.

### Results

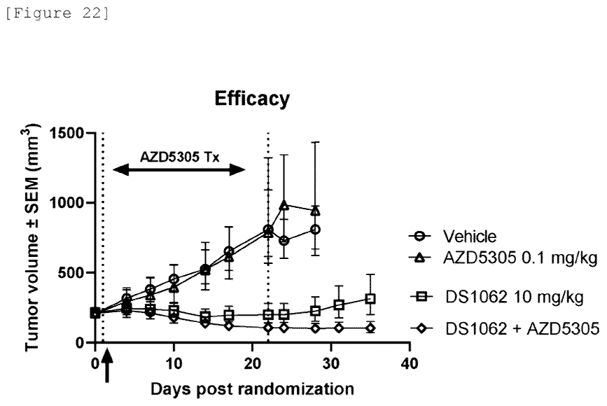

Tumor volumes for treatments with DS-1062 or AZD5305 alone or with DS-1062 in combination with AZD5305 are shown in FIG. 21. Data represents change in tumor volume over time for treatment groups. The dotted lines in FIG. 21 represent start and end of AZD5305 dosing cycles. For full dose and schedule information, refer to Table 12 above. Values shown are mean±SEM; n=8 for all treatment groups.

TGI responses (Day 52 TGI %) following treatment with DS-1062 or AZD5305 alone or with DS-1062 in combination with AZD5305, in NCI-N87 xenograft, are shown in Table 13:

TABLE 13

| | TGI % | p-value vs | |
|---|---|---|---|
| Treatment group | day 52 | vehicle | Significance |
| DS-1062 10 mg/kg | 84% | 0.0001 | **** |
| AZD5305 1 mg/kg | 26% | 0.1222 | †ns |
| DS-1062 10 mg/kg + | 95% | 0.0001 | **** |
| AZD5305 0.1 mg/kg | | | |
| (Day 1-21) | | | |
| DS-1062 10 mg/kg + | 93% | 0.0001 | **** |
| AZD5305 0.1 mg/kg | | | |
| (Day 1-7) | | | |
| DS-1062 10 mg/kg + | 89% | 0.0001 | **** |
| AZD5305 0.1 mg/kg | | | |
| (Day 8-15) | | | |
| DS-1062 10 mg/kg + | 91% | 0.0001 | **** |
| AZD5305 0.03 mg/kg | | | |
| (Day 8-15) | | | |

†not significant

Monotherapy with DS-1062 at 10 mg/kg showed TGI value of 84% at day 52 post treatment. AZD5305 monotherapy achieved a TGI of 26% at day 52 post treatment. Combination treatment of DS-1062 with AZD5305 at 0.1 mg/kg from Day 1-21 resulted in a TGI of 95% at 52 days post treatment, combination with AZD5305 at 0.1 mg/kg from Day 1-7 resulted in a TGI of 93% at 52 days post treatment, combination with AZD5305 at 0.1 mg/kg from Day 8-15 resulted in a TGI of 89% at 52 days post treatment, and combination with AZD5305 at 0.03 mg/kg from Day 8-15 resulted in a TGI of 918, indicating that reducing the dose scheduling of AZD5305 from 21 days to 7 days does not lower efficacy of this combination.

### Example 8: Antitumor Test—In Vivo—CTG-3718 Xenograft Model

Combination of antibody-drug conjugate DS-1062 (datopotamab deruxtecan) with PARP1 selective inhibitor AZD5305 (5-[4-[(7-ethyl-6-oxo-5H-1,5-naphthyridin-3-yl)methyl]piperazin-1-yl]-N-methyl-pyridine-2-carboxamide)

### Method:

The human patient-derived xenograft (PDX) model, CTG-3718, was established from fragments of freshly resected tumor of an ovarian cancer patient who relapsed on treatment with PARP inhibitor talazoparib. This PDX was obtained in accordance with appropriate consent procedures. This ovarian PDX model was subcutaneously passaged in vivo as fragments from animal to animal in nude mice. When tumors reached approximately 250 mm³, similar-sized tumors were randomly assigned to treatment groups as shown in Table 14:

TABLE 14

| | | | Route of | Dosing Schedule |
|---|---|---|---|---|
| Treatment | Dose | | administration | (21 days) |
| Vehicle | — | | IV + PO | Single dose + QD |
| DS-1062 | 10 | mg/kg | IV | Single dose |
| AZD5305 | 0.1 | mg/kg | PO | QD |

| | | | |
|---|---|---|---|
| DS-1062 + | 10 mg/kg + | IV + PO | Single dose + QD |
| AZD5305 | 0.1 mg/kg | | |

PO: oral (per os) dosing
QD: once per day (quaque die) dosing

### Results

Tumor volumes for treatments with DS-1062 or AZD5305 alone or with DS-1062 in combination with AZD5305 are shown in FIG. 22. Data represents change in tumor volume over time for treatment groups. The dotted lines in FIG. 22 represent start and end of AZD5305 dosing. For full dose and schedule information, refer to Table 14 above. Values shown are mean±SEM; n=8 for all treatment groups.

TGI responses (Day 28 TGI %) following treatment with DS-1062 or AZD5305 alone or with DS-1062 in combination with AZD5305, in CTG-3718 xenograft, are shown in Table 15:

TABLE 15

| | TGI % | p-value vs | |
|---|---|---|---|
| Treatment group | day 28 | vehicle | Significance |
| DS-1062 10 mg/kg | 72% | 0.0008 | *** |
| AZD5305 0.1 mg/kg | −16% | 0.9999 | †ns |
| DS-1062 10 mg/kg + | 88% | 0.0001 | **** |
| AZD5305 0.1 mg/kg | | | |

†not significant

Monotherapy with DS-1062 at 10 mg/kg showed TGI value of 72% at day 28 post treatment. AZD5305 monotherapy at 0.1 mg/kg achieved a TGI of −16% at day 28 post treatment. Combination treatment of DS-1062 with AZD5305 at 0.1 mg/kg from resulted in a TGI of 88% at 28 days post treatment indicating that the combination displayed higher efficacy than either monotherapy.

The foregoing written specification is considered to be sufficient to enable one skilled in the art to practice the embodiments. The foregoing description and

Examples detail certain embodiments and describe the best mode contemplated by the inventors. It will be appreciated, however, that no matter how detailed the foregoing may appear in text, the embodiments may be practiced in many ways and the claims include any equivalents thereof.

### Free Text of Sequence Listing

SEQ ID NO: 1-Amino acid sequence of a heavy chain of anti-TROP2 antibody
SEQ ID NO: 2-Amino acid sequence of a light chain of anti-TROP2 antibody
SEQ ID NO: 3-Amino acid sequence of a heavy chain CDRH1 [=amino acid residues 50 to 54 of SEQ ID NO: 1]
SEQ ID NO: 4-Amino acid sequence of a heavy chain CDRH2 [=amino acid residues 69 to 85 of SEQ ID NO: 1]
SEQ ID NO: 5-Amino acid sequence of a heavy chain CDRH3 [=amino acid residues 118 to 129 of SEQ ID NO: 1]
SEQ ID NO: 6-Amino acid sequence of a light chain CDRL1 [=amino acid residues 44 to 54 of SEQ ID NO: 2]
SEQ ID NO: 7-Amino acid sequence of a light chain CDRL2 [=amino acid residues 70 to 76 of SEQ ID NO: 2]
SEQ ID NO: 8-Amino acid sequence of a light chain CDRL3 [=amino acid residues 109 to 117 of SEQ ID NO: 2]
SEQ ID NO: 9-Amino acid sequence of a heavy chain variable region [=amino acid residues 20 to 140 of SEQ ID NO: 1]
SEO ID NO: 10-Amino acid sequence of a light chain variable region [=amino acid residues 21 to 129 of SEQ ID NO: 2]
SEQ ID NO: 11-Amino acid sequence of a heavy chain [=amino acid residues 20 to 469 of SEQ ID NO: 1]
SEQ ID NO: 12-Amino acid sequence of a heavy chain [=amino acid residues 20 to 470 of SEQ ID NO: 1]
SEQ ID NO: 13-Amino acid sequence of a light chain [=amino acid residues 21 to 234 of SEQ ID NO: 2]

1. A pharmaceutical product comprising an anti-TROP2 antibody-drug conjugate and a PARP1 selective inhibitor for administration in combination, wherein the anti-TROP2 antibody-drug conjugate is an antibody-drug conjugate in which a drug-linker represented by the following formula: 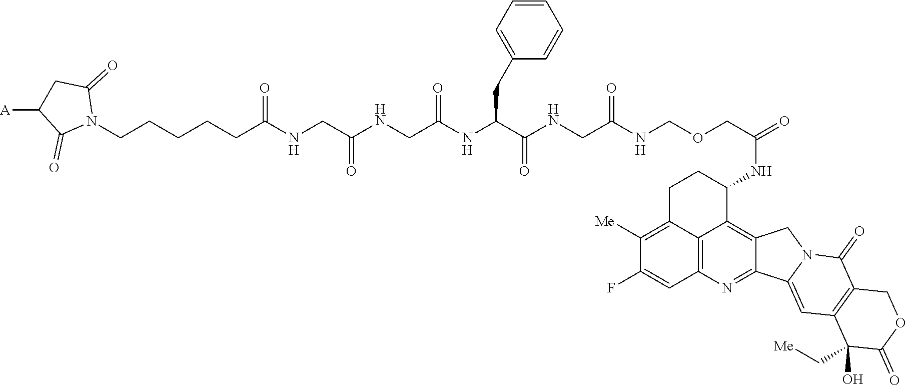
wherein A represents the connecting position to an antibody, is conjugated to an anti-TROP2 antibody via a thioether bond.

2. The pharmaceutical product according to claim 1, wherein the PARP1 selective inhibitor is a compound represented by the following formula (I): 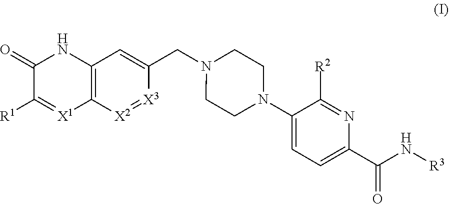
wherein:
X¹ and X² are each independently selected from N and C(H),
X³ is independently selected from N and C(R⁴), wherein R⁴ is H or fluoro,
R¹ is C₁₋₄ alkyl or C₁₋₄ fluoroalkyl,
R² is independently selected from H, halo, C₁₋₄ alkyl, and C₁₋₄ fluoroalkyl, and
R³ is H or C₁₋₄ alkyl,
or a pharmaceutically acceptable salt thereof
provided that:
when X¹ is N, then X² is C(H), and X³ is C(R⁴),
when X² is N, then X¹═C(H), and X³ is C(R⁴), and
when X³ is N, then X¹ and X² are both C(H).

3. The pharmaceutical product according to claim 2 wherein, in formula (I), R³ is C₁₋₄ alkyl.

4. The pharmaceutical product according to claim 3 wherein, in formula (I), R³ is methyl.

5. The pharmaceutical product according to claim 2 wherein, in formula (I), R¹ is ethyl.

6. The pharmaceutical product according to claim 1, wherein the PARP1 selective inhibitor is a compound represented by the following formula (Ia): 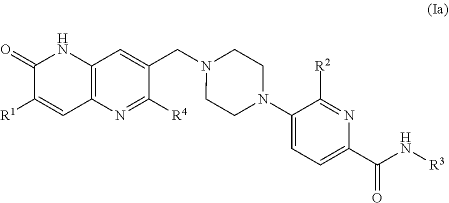
wherein
R¹ is C₁₋₄ alkyl,
R² is selected from H, halo, C₁₋₄ alkyl, and C₁₋₄ fluoroalkyl,
R³ is H or C₁₋₄ alkyl, and
R⁴ is H,
or a pharmaceutically acceptable salt thereof.

7. The pharmaceutical product according to claim 6 wherein, in formula (Ia), R² is H or halo.

8. The pharmaceutical product according to claim 6 wherein in formula (Ia), R¹ is ethyl, R² is selected from H, chloro and fluoro, and R³ is methyl.

9. The pharmaceutical product according to claim 1, wherein the PARP1 selective inhibitor is AZD5305, also known as AZ14170049, represented by the following formula: 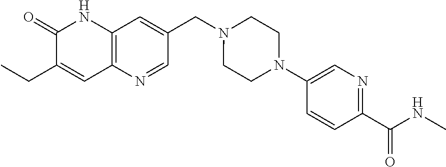 [SMILES: CCc1cc2ncc(CN3CCN(c4ccc(C(=O)NC)nc4)CC3)cc2[nH]c1=O]
or a pharmaceutically acceptable salt thereof.

10. The pharmaceutical product according to claim 1, wherein the anti-TROP2 antibody is an antibody comprising a heavy chain comprising CDRH1 consisting of an amino acid sequence represented by SEQ ID NO: 3, CDRH2 consisting of an amino acid sequence represented by SEQ ID NO: 4 and CDRH3 consisting of an amino acid sequence represented by SEQ ID NO: 5, and a light chain comprising CDRL1 consisting of an amino acid sequence represented by SEQ ID NO: 6, CDRL2 consisting of an amino acid sequence represented by SEQ ID NO: 7 and CDRL3 consisting of an amino acid sequence represented by SEQ ID NO: 8.

11. The pharmaceutical product according to claim 10, wherein the anti-TROP2 antibody is an antibody comprising a heavy chain comprising a heavy chain variable region consisting of an amino acid sequence represented by SEQ ID NO: 9 and a light chain comprising a light chain variable region consisting of an amino acid sequence represented by SEQ ID NO: 10.

12. The pharmaceutical product according to claim 11, wherein the anti-TROP2 antibody is an antibody comprising a heavy chain consisting of an amino acid sequence represented by SEQ ID NO: 12 and a light chain consisting of an amino acid sequence represented by SEQ ID NO: 13.

13. The pharmaceutical product according to claim 11, wherein the anti-TROP2 antibody is an antibody comprising a heavy chain consisting of an amino acid sequence represented by SEQ ID NO: 11 and a light chain consisting of an amino acid sequence represented by SEQ ID NO: 13.

14. The pharmaceutical product according to claim 1, wherein the average number of units of the drug-linker conjugated per antibody molecule in the antibody-drug conjugate is in the range of from 2 to 8.

15. The pharmaceutical product according to claim 14, wherein the average number of units of the drug-linker conjugated per antibody molecule in the antibody-drug conjugate is in the range of from 3.5 to 4.5.

16. The pharmaceutical product according to claim 15, wherein the anti-TROP2 antibody-drug conjugate is datopotamab deruxtecan (DS-1062).

17. The pharmaceutical product according to claim 1 wherein the product is a composition comprising the anti-TROP2 antibody-drug conjugate and the PARP1 selective inhibitor, for simultaneous administration.

18. The pharmaceutical product according to claim 1 wherein the product is a combined preparation comprising the anti-TROP2 antibody-drug conjugate and the PARP1 selective inhibitor, for sequential or simultaneous administration.

19. The pharmaceutical product according to claim 1 wherein the anti-TROP2 antibody-drug conjugate is to be administered at a dose of 6 mg/kg body weight.

20. The pharmaceutical product according to claim 19 wherein the dose of the anti-TROP2 antibody-drug conjugate is to be administered once every three weeks.

21. The pharmaceutical product according to claim 1 wherein the PARP1 selective inhibitor is to be administered daily for the first week, second week, and/or third week of a three week cycle.

22-76. (canceled)

77. A method of treating cancer comprising administering an anti-TROP2 antibody-drug conjugate and a PARP1 selective inhibitor as defined in claim 1 in combination to a subject in need thereof.

78. The method according to claim 77, wherein the cancer is as at least one selected from the group consisting of breast cancer, lung cancer, colorectal cancer, gastric cancer, esophageal cancer, head-and-neck cancer, esophagogastric junction adenocarcinoma, biliary tract cancer, Paget's disease, pancreatic cancer, ovarian cancer, uterine carcinosarcoma, urothelial cancer, prostate cancer, bladder cancer, gastrointestinal stromal tumor, digestive tract stromal tumor, cervical cancer, squamous cell carcinoma, peritoneal cancer, liver cancer, hepatocellular cancer, corpus uteri carcinoma, kidney cancer, vulval cancer, thyroid cancer, penis cancer, leukemia, malignant lymphoma, plasmacytoma, myeloma, glioblastoma multiforme, osteosarcoma, sarcoma, endometrial cancer and melanoma.

79. The method according to claim 77, wherein the method comprises administering the anti-TROP2 antibody-drug conjugate and the PARP1 selective inhibitor sequentially.

80. The method according to claim 77 wherein the method comprises administering the anti-TROP2 antibody-drug conjugate at a dose of 6 mg/kg body weight.

81. The method according to claim 80 wherein the method comprises administering the dose of the anti-TROP2 antibody-drug conjugate once every three weeks.

82. The method according to claim 77 wherein the method comprises administering the PARP1 selective inhibitor daily for the first week, second week, and/or third week of a three week cycle.

83. The method according to claim 77, wherein the method comprises administering the anti-TROP2 antibody-drug conjugate and the PARP1 selective inhibitor simultaneously.

84. The method according to claim 78, wherein the cancer is deficient in Homologous Recombination (HR) dependent DNA DSB repair activity.

85. The method according to claim 78, wherein the cancer is not deficient in Homologous Recombination (HR) dependent DNA DSB repair activity.

86. The method according to claim 78, wherein the cancer is exhibits resistance or refractoriness to a previous treatment with a PARP inhibitor.

87. The method product according to claim 86, wherein the previous treatment is with a PARP inhibitor selected from olaparib, rucaparib, niraparib, talazoparib and veliparib.


SEQUENCE LISTING (13 sequences)

SEQ ID NO: 1 - protein, 470 aa, synthetic construct
  REGION 1..470: note = Heavy chain of anti-TROP2 antibody
MKHLWFFLLLVAAPRWVLSQVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 234 aa, synthetic construct
  REGION 1..234: note = Light chain of anti-TROP2 antibody
MVLQTQVFISLLLWISGAYGDIQMTQSPSSLSASVGDRVTITCKASQDVSTAVAWYQQKPGKAPKLLIYSASYRYTGVPSRFSGSGSGTDFTLTISSLQPEDFAVYYCQQHYITPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 5 aa, synthetic construct
  REGION 1..5: note = CDRH1 of heavy chain of anti-TROP2 antibody
TAGMQ

SEQ ID NO: 4 - protein, 17 aa, synthetic construct
  REGION 1..17: note = CDRH2 of heavy chain of anti-TROP2 antibody
WINTHSGVPKYAEDFKG

SEQ ID NO: 5 - protein, 12 aa, synthetic construct
  REGION 1..12: note = CDRH3 of heavy chain of anti-TROP2 antibody
SGFGSSYWYFDV

SEQ ID NO: 6 - protein, 11 aa, synthetic construct
  REGION 1..11: note = CDRL1 of light chain of anti-TROP2 antibody
KASQDVSTAVA

SEQ ID NO: 7 - protein, 7 aa, synthetic construct
  REGION 1..7: note = CDRL2 of light chain of anti-TROP2 antibody
SASYRYT

SEQ ID NO: 8 - protein, 9 aa, synthetic construct
  REGION 1..9: note = CDRL3 of light chain of anti-TROP2 antibody
QQHYITPLT

SEQ ID NO: 9 - protein, 121 aa, synthetic construct
  REGION 1..121: note = Variable region of heavy chain of anti-TROP2 antibody
QVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSSYWYFDVWGQGTLVTVSS

SEQ ID NO: 10 - protein, 109 aa, synthetic construct
  REGION 1..109: note = Variable region of light chain of anti-TROP2 antibody
DIQMTQSPSSLSASVGDRVTITCKASQDVSTAVAWYQQKPGKAPKLLIYSASYRYTGVPSRFSGSGSGTDFTLTISSLQPEDFAVYYCQQHYITPLTFGQGTKLEIKRT

SEQ ID NO: 11 - protein, 450 aa, synthetic construct
  REGION 1..450: note = Heavy chain of anti-TROP2 antibody
QVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 12 - protein, 451 aa, synthetic construct
  REGION 1..451: note = Heavy chain of anti-TROP2 antibody
QVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 214 aa, synthetic construct
  REGION 1..214: note = Light chain of anti-TROP2 antibody
DIQMTQSPSSLSASVGDRVTITCKASQDVSTAVAWYQQKPGKAPKLLIYSASYRYTGVPSRFSGSGSGTDFTLTISSLQPEDFAVYYCQQHYITPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC